(19) United States
(12) Patent Application Publication
(10) Pub. No.: US 2018/0264146 A1
(43) Pub. Date: Sep. 20, 2018

(54) NOVEL IMAGING COMPOUNDS

(71) Applicants: AC IMMUNE S.A., Lausanne (CH); PIRAMAL IMAGING SA, Matran (CH)

(72) Inventors: Heiko KROTH, Ecublens (CH); Sreenivasachary NAMPALLY, Ecublens (CH); Jérôme MOLETTE, Prevessin Moens (FR); Emanuele GABELLIERI, Lausanne (CH); Hanno SCHIEFERSTEIN, Berlin (DE); Andre MÜLLER, Berlin (DE); Heribert SCHMITT-WILLICH, Berlin (DE); Mathias BERNDT, Berlin (DE)

(73) Assignees: AC IMMUNE S.A., Lausanne (CH); PIRAMAL IMAGING SA, Matran (CH)

(21) Appl. No.: 15/745,042

(22) PCT Filed: Jul. 15, 2016
(86) PCT No.: PCT/EP2016/066901

§ 371 Date: Jan. 15, 2018

(30) Foreign Application Priority Data
Jul. 15, 2015 (EP) 15176938.7

(51) Int. Cl. A61K 51/04

(52) U.S. Cl. CPC A61K 51/0455; C07D 471/14; A61K 2123/00

(57) ABSTRACT

The present invention relates to novel compounds that can be employed in the selective Tau detection of disorders and abnormalities associated with Tau aggregates such as Alzheimer's disease and other tauopathies using Positron Emission Tomography (PET) Imaging.

### FIELD OF THE INVENTION

The present invention relates to novel compounds that can be employed in the selective detection of disorders and abnormalities associated with Tau aggregates such as Alzheimer's disease and other tauopathies, for example, using Positron Emission Tomography (PET) Imaging.

### BACKGROUND OF THE INVENTION

Alzheimer's disease (AD) is a neurological disorder primarily thought to be caused by amyloid plaques, an extracellular accumulation of abnormal deposit of A13 aggregates in the brain. The other major neuropathological hallmarks in AD are the intracellular neurofibrillary tangles (NFT) that originate by the aggregation of the hyperphosphoryiated Tau (Tubulin associated unit) protein, phosphorylated Tau or pathological Tau and its conformers. AD shares this pathology with many neurodegenerative tauopathies, in particularly with specified types of frontotemporal dementia (FTD). In AD brain, Tau pathology (tauopathy) develops later than amyloid pathology, but it is still discussed controversially if amyloid ß protein is the causative agent in AD which constitutes the essence of the so-called amyloid cascade hypothesis (Hardy et al, Science 1992, 256, 184-185, and most recently, Musiek et al, Nature Neurosciences 2015, 18(6), 800-806): “Three dimensions of the amyloid hypothesis: time, space and Wingmen”.

Presently, the only definite way to diagnose AD is to identify plaques and tangles in brain tissue by histological analysis of biopsy or autopsy materials after the death of the individual.

Therefore, there is a great deal of interest in detection of Tau pathology in vivo. Tau PET Imaging promises novel insights into deposition of Tau aggregates in the human brain and might allow to non-invasively examine the degree of Tau pathology, quantify changes in Tau deposition over time, assess its correlation with cognition and analyze the efficacy of an anti-Tau therapy. For recent reviews see Shah et al, J Nucl Med. 2014, 55(6), 871-874: “Molecular Imaging Insights into Neurodegeneration: Focus on Tau PET Radiotracers” and Ariza et al, J Med Chem 2015, 58(11), 4365-82): “Tau PET Imaging: Past, Present and Future”. In addition, several patent applications have recently been published, e.g: WO 2009/102498 and WO 2011/119565 (both Siemens Medical Solutions), WO 2012/067863 and WO 2012/068072 (both GE Healthcare), WO 2015/052105 (Hoffmann-La Roche AG) which claim novel compounds for Tau Imaging.

In order to achieve high target selectivity, molecular probes have been used which recognize and bind to the pathological target. Selectivity for binding to pathological Tau protein over other biological entities is therefore a basic requirement of an imaging probe. In order to reduce background signal interference resulting from non-specific off-target binding (e.g. binding to amyloid-beta or monoamine oxidases), and to reduce dosing requirements, imaging compounds should bind with high affinity to their target. Since amyloid or amyloid-like deposits formed from proteins of diverse primary amino acid sequences share a common β-sheet quaternary conformation, molecular probes are required that can differentiate such structures in order to avoid detection of false-positives and misdiagnosis.

In addition, molecular probes must also be designed such that upon administration they can distribute within the body and reach their target. For imaging of Tau aggregates associated with neurological disorders such as e.g. Alzheimer's disease, imaging compounds are required that can penetrate the blood brain barrier and pass into the relevant regions of the brain. For targeting intracellular Tau aggregates, cell permeability is a further requirement of imaging compounds. A further prerequisite in order to avoid unnecessary accumulation of compound which may result in increased risk of unwanted side-effects, is a fast compound wash-out from the brain (or other targeting organ).

The compounds of the present invention demonstrate high affinity to Tau aggregates, high selectivity against other targets in the brain and favorable pharmacokinetic properties.

### DESCRIPTION OF THE FIGURES

FIG. 1 shows the ex vivo autoradiograph on human brain sections with ¹⁸F-2. a)¹⁸F-2 without competition, b)¹⁸F-2 with an excess of non-radioactive 2, c) competition of ¹⁸F-2 with amyloid-beta binder.

FIG. 2 shows the ex vivo autoradiograph on human brain sections with ¹⁸F-1. a)¹⁸F-1 without competition, b)¹⁸F-1 with an excess of non-radioactive 1, c) competition of ¹⁸F-1 with amyloid-beta binder.

FIG. 3 shows the washout kinetics of the compound ¹⁸F-1.

### SUMMARY OF THE INVENTION

The present invention relates to compounds of general formula (I)

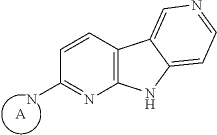

and all stereoisomers and mixtures thereof, pharmaceutically acceptable salts, hydrates, solvates and polymorphs thereof;

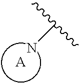

is selected from a 5- to 7-membered monocyclic group and a 7- to 9-membered bicyclic group, which can optionally contain a heteroatom selected from N and O in addition to the nitrogen atom which is attached to the tricyclic group and wherein

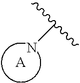

is substituted by a substituent R and is optionally substituted by a substituent R*. Preferably,

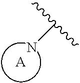

is selected from

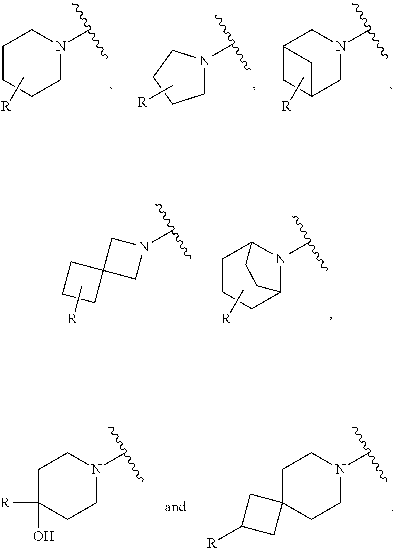

R is selected from F—, alkylene which is substituted by F, (alkylene-oxy)ₙ- which is substituted by F, (alkylene-oxy)ₙ-alkylene-which is substituted by F, and alkylene-oxy which is substituted by F and OH.

Within the present invention it is understood that the term “substituted by . . . ” means that one or more of the respective substituents is present. Thus, for example, the expression “alkylene which is substituted by F” means that the alkylene group is substituted by one or more F.

In one embodiment, preferred examples of R include F—, F—CH₂CH₂OCH₂CH₂—, F-CD₂CH₂CH₂O—, F—CH₂CH₂O—, F—(CH₂CH₂O)₂—, F—CH₂CH₂O—CH₂—, F—(CH₂CH₂O)₂—CH₂—, F—CH₂CH₂CH₂O—, F—CH₂CH(OH)CH₂O—, F—CH₂CH(F)CH₂O—, HO—CH₂CH(F)CH₂O— and F—CH₂—CH(CH₂OH)—O; more preferably R is selected from F, F—CH₂CH₂OCH₂CH₂—, F-CD₂CH₂CH₂O—, F—CH₂CH₂O—, F—(CH₂CH₂O)₂—, F—CH₂CH(OH)CH₂—O—, F—CH₂CH(F)CH₂O—, and HO—CH₂CH(F)CH₂—O—.

If R is attached to a N-atom, R is preferably selected from F—CH₂CH₂—, F—CH₂CH₂OCH₂CH₂—, F—CH₂CH₂CH₂—, F—CH₂CH(OH)CH₂— and HO—CH₂CH(F)CH₂—.

An example of a preferred alkylene-oxy which is substituted by F and OH is

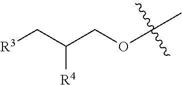

wherein R³ is OH or F and wherein R⁴ is OH or F, with the proviso that if R³ is OH, R⁴ is F, or if R³ is F, R⁴ is OH.

Another example of a preferred alkylene-oxy which is substituted by F and OH or F only is

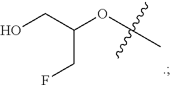 [SMILES: *OC(CO)CF]

R* is an optional substituent which is selected from hydroxy, methoxy, methyl and hydroxymethyl.

n is 1, 2 or 3.

Fluoro is [F-19]fluoro or [F-18]fluoro, preferably [F-18]fluoro.

In one embodiment, the invention refers to a compound of formula (IA):

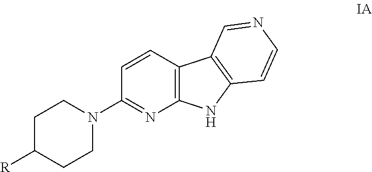

wherein R is as defined above.

In one embodiment, the invention refers to a compound of formula (IB):

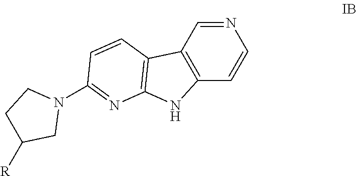

wherein R is as defined above.

In one embodiment, the in refers to a compound of formula (IC):

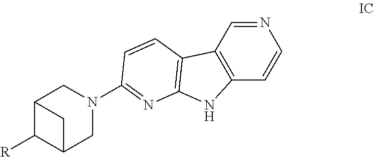

wherein R is as defined above.

In one embodiment, the invention refers to a compound of formula (ID):

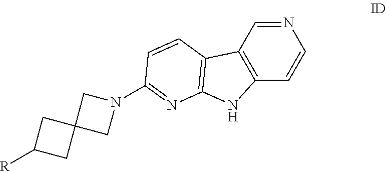

wherein R is as defined above.

In one embodiment, the invention refers to a compound of formula (IE):

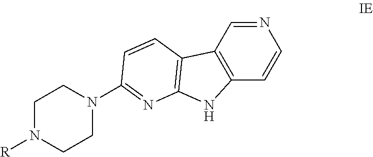

wherein R is as defined above.

In one embodiment, the invention refers to a compound of formula (IF):

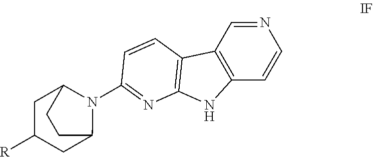

wherein R is as defined above.

In one embodiment, the invention refers to a compound of formula (IG):

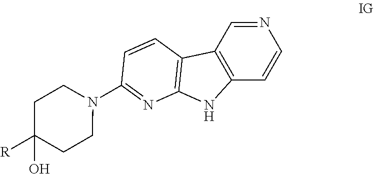

wherein R is as defined above.

In one embodiment, the invention refers to a compound of formula (IH):

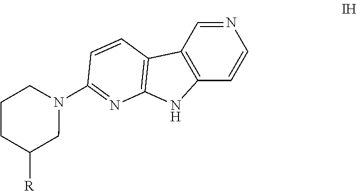

wherein R is as defined above.

In one embodiment, the invention refers to a compound of formula (IJ):

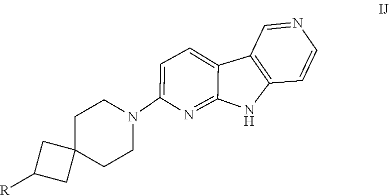

wherein R is as defined above.

The compounds of formula (IA), (IB), (IC), (ID), (IE), (IF), (IG), (IH) and (IJ) can be substituted by R*, if desired.

Examples thereof include

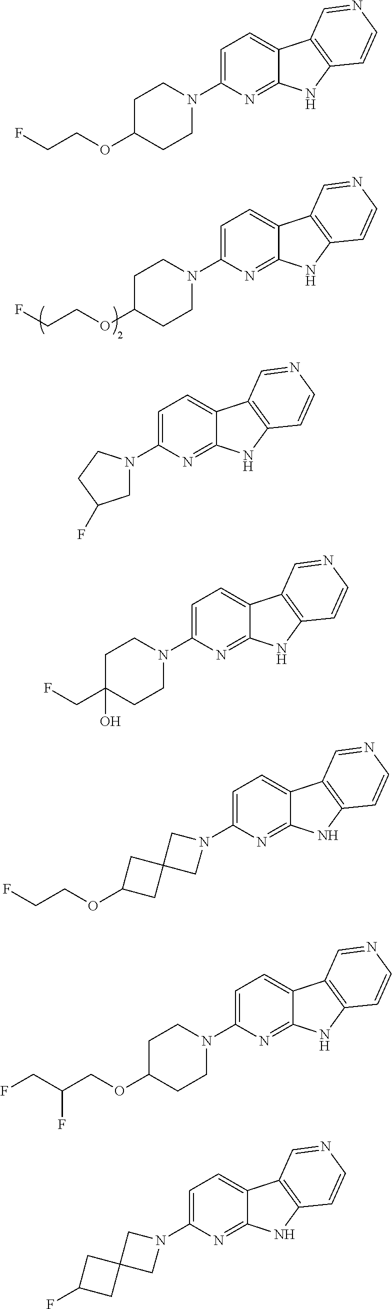

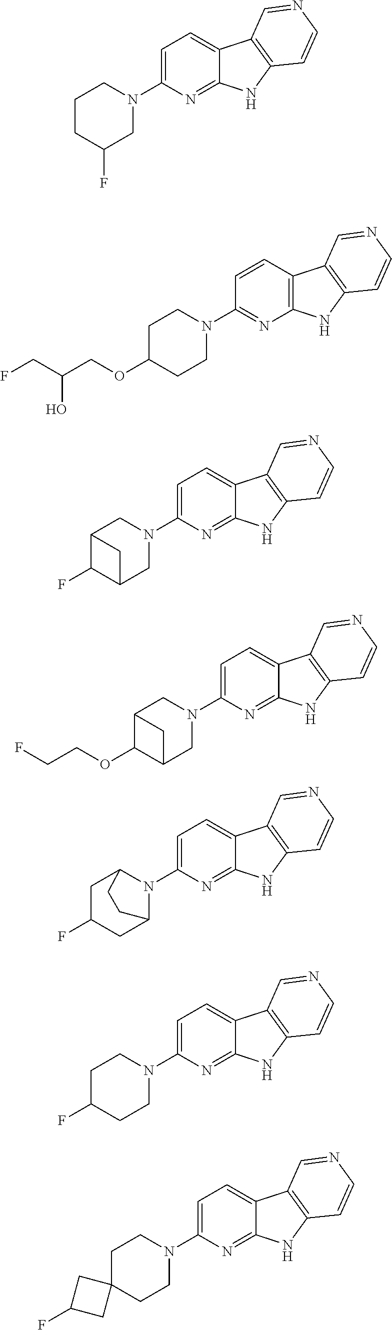

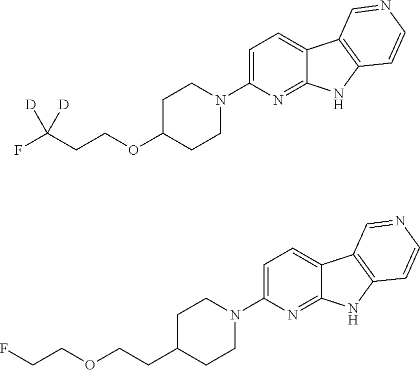 [SMILES: [2H]C([2H])(F)CCOC1CCN(c2ccc3c(n2)[nH]c2ccncc23)CC1 ; FCCOCCC1CCN(c2ccc3c(n2)[nH]c2ccncc23)CC1]

The present invention also provides a compound of formula (II):

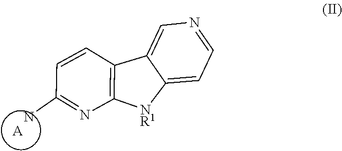

and all stereoisomers and mixtures thereof, pharmaceutically acceptable salts, hydrates, solvates and polymorphs thereof;

which is a precursor of the compound of formula (I).

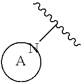

is selected from a 5- to 7-membered monocyclic group and a 7- to 9-membered bicyclic group, which can optionally contain a heteroatom selected from N and O in addition to the nitrogen atom which is attached to the tricyclic group and wherein

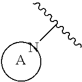

is substituted by a substituent R^{p} and is optionally substituted by a substituent R**.

Preferably,

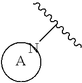

is selected from

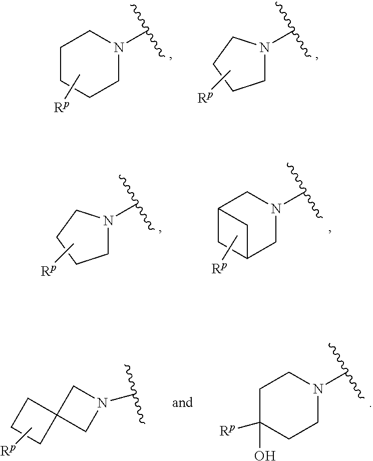

In one embodiment, preferred examples of R^{p} include LG-, LG-CH₂CH₂O—, LG-(CH₂CH₂O)₂—, LG-CH₂CH₂O—CH₂—, LG-(CH₂CH₂O)—CH₂—, LG-CH₂CH₂CH₂O—, LG-CH₂CH(OPG²)CH₂O—, PG²O—CH₂CH(LG)CH₂O— and LG-CH₂—CH(CH₂OPG²)-O; more preferably R^{p} is selected from LG-, LG-CH₂CH₂O—, LG-(CH₂CH₂O)₂—, LG-CH₂CH(OPG²)CH₂—O— and PG²O—CH₂CH(LG)CH₂—O—.

If R^{p} is attached to a N-atom, R^{p} is preferably selected from LG-CH₂CH₂—, LG-CH₂CH₂OCH₂CH₂—, LG-CH₂CH₂CH₂—, LG-CH₂CH(OPG²)CH₂— and PG²O—CH₂CH(LG)CH₂—.

An example of a preferred alkylene-oxy which is substituted by LG and OPG² is

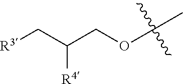

wherein R^{3′} is OPG² or LG and wherein R^{4′} is OPG² or LG, with the proviso that if R^{3′} is OPG², R^{4′} is LG, or if R^{3′} is LG, R^{4′} is OPG².

Another example of a preferred alkylene-oxy which is substituted by LG and OPG² is

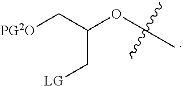

R** is an optional substituent which is selected from hydroxy, methoxy, methyl and hydroxymethyl.

n is 1, 2 or 3.

R¹ is —H or PG¹, wherein PG¹ is an amine protecting group. Examples of the amine protecting group include tert-butyloxycarbonyl (Boc), p-tolylsulfonyl (Tosyl), trimethylsilylethoxycarbonyl (Teoc), carbobenzyloxy (Cbz), p-methoxybenzyl carbonyl (Moz or MeOZ), 9-fluorenylmethyloxycarbonyl (Fmoc), acetyl (Ac), triphenylmethyl (Trt) or trifluoroacetyl. Preferred examples are (CH₃)₃C—OCO—, (C₆H₅)₃C— and CH₃C₆H₄—SO₂—. In one embodiment R¹ is —H. In another embodiment R¹ is PG¹.

PG² is a hydroxy protecting group, preferably

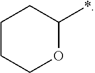

LG is a leaving group.

In one preferred embodiment, the present invention relates to compounds of formula (IIA):

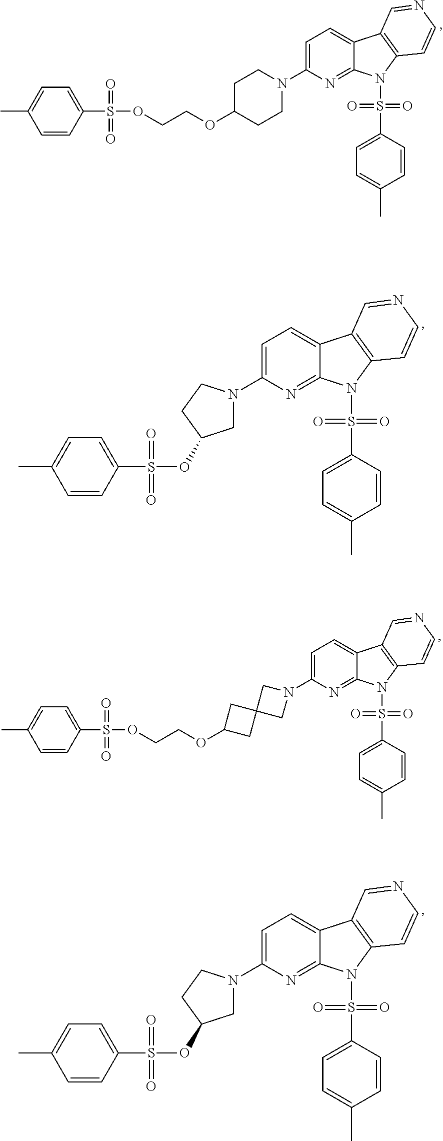 [SMILES: Cc1ccc(S(=O)(=O)OCCOC2CCN(c3ccc4c5cnccc5n(S(=O)(=O)c5ccc(C)cc5)c4n3)CC2)cc1 ; Cc1ccc(S(=O)(=O)O[C@@H]2CCN(c3ccc4c5cnccc5n(S(=O)(=O)c5ccc(C)cc5)c4n3)C2)cc1 ; Cc1ccc(S(=O)(=O)OCCOC2CC3(C2)CN(c2ccc4c5cnccc5n(S(=O)(=O)c5ccc(C)cc5)c4n2)C3)cc1 ; Cc1ccc(S(=O)(=O)O[C@H]2CCN(c3ccc4c5cnccc5n(S(=O)(=O)c5ccc(C)cc5)c4n3)C2)cc1]

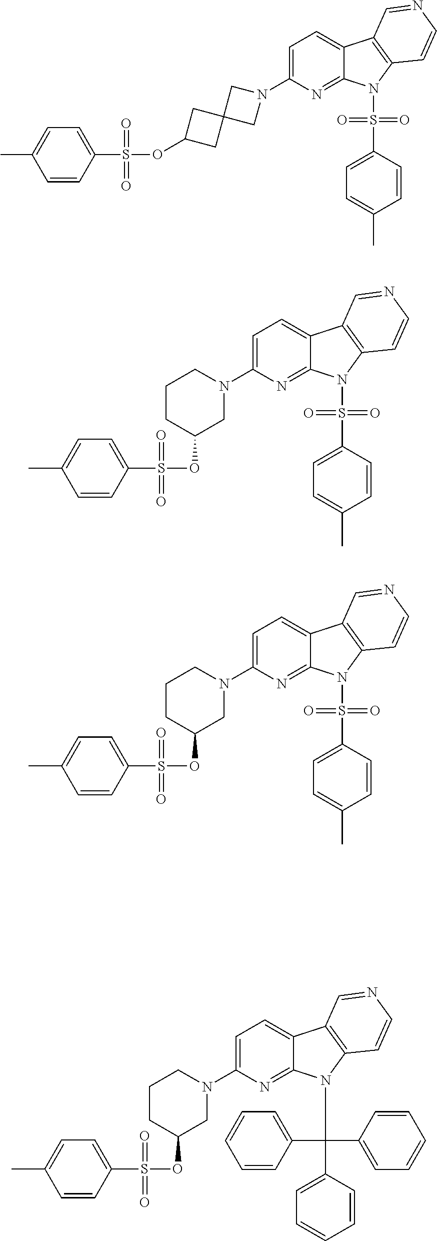 [SMILES: Cc1ccc(S(=O)(=O)OC2CC3(C2)CN(c2ccc4c5cnccc5n(S(=O)(=O)c5ccc(C)cc5)c4n2)C3)cc1 ; Cc1ccc(S(=O)(=O)O[C@@H]2CCCN(c3ccc4c5cnccc5n(S(=O)(=O)c5ccc(C)cc5)c4n3)C2)cc1 ; Cc1ccc(S(=O)(=O)O[C@H]2CCCN(c3ccc4c5cnccc5n(S(=O)(=O)c5ccc(C)cc5)c4n3)C2)cc1 ; Cc1ccc(S(=O)(=O)O[C@H]2CCCN(c3ccc4c5cnccc5n(C(c5ccccc5)(c5ccccc5)c5ccccc5)c4n3)C2)cc1]

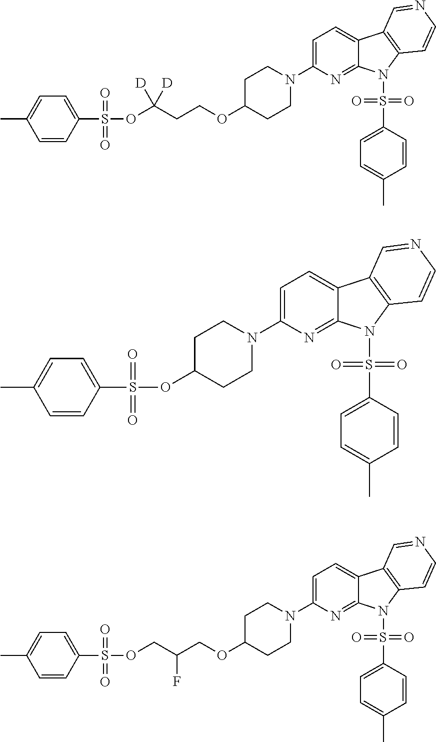 [SMILES: [2H]C([2H])(CCOC1CCN(c2ccc3c4cnccc4n(S(=O)(=O)c4ccc(C)cc4)c3n2)CC1)OS(=O)(=O)c1ccc(C)cc1 ; Cc1ccc(S(=O)(=O)OC2CCN(c3ccc4c5cnccc5n(S(=O)(=O)c5ccc(C)cc5)c4n3)CC2)cc1 ; Cc1ccc(S(=O)(=O)OCC(F)COC2CCN(c3ccc4c5cnccc5n(S(=O)(=O)c5ccc(C)cc5)c4n3)CC2)cc1]

The compounds of formula (IIA) can be substituted by R**, if desired.

It is understood that all combinations of the above mentioned embodiments are also envisaged.

The compounds of the present invention can be detectably labeled. The type of the label is not specifically limited and will depend on the detection method chosen. Examples of possible labels include isotopes such as radionuclides, positron emitters, gamma emitters, as well as fluorescent, luminescent and chromogenic labels. With respect to the detectably labeled compounds of the present invention which include a radioisotope, a positron emitter, or a gamma emitter, it is to be understood that the radioisotope, positron emitter, or gamma emitter is to be present in an amount which is not identical to the natural amount of the respective radioisotope, positron emitter, or gamma emitter. Furthermore, the employed amount should allow detection thereof by the chosen detection method.

Examples of suitable isotopes such as radionuclides, positron emitters and gamma emitters include ²H, ³H, ¹⁸F, ¹²³I, ¹²⁴I, ¹²⁵I, ¹³¹I, ¹¹C, ¹³N, ¹⁵O, and ⁷⁷Br, preferably ²H, ³H, ¹¹C, ¹³N, ¹⁵O, and ¹⁸F, more preferably ²H, ³H and ¹⁸F, even more preferably ¹⁸F.

¹⁸F-labeled compounds are particularly suitable for imaging applications such as PET. The corresponding compounds which include fluorine having a natural ¹⁹F isotope are also of particular interest as they can be used as analytical standards and references during manufacturing, quality control, release and clinical use of their ¹⁸F-analogs.

Further, substitution with isotopes such as deuterium, i.e., ²H, may afford certain diagnostic advantages resulting from greater metabolic stability by reducing for example defluorination, increased in vivo half-life or reduced dosage requirements, while keeping or improving the original compound efficacy.

Isotopic variations of the compounds of the invention can generally be prepared by conventional procedures such as by the illustrative methods or by the preparations described in the Examples and Preparative Examples hereafter using appropriate isotopic variations of suitable reagents, commercially available or prepared by known synthetic techniques.

Radionuclides, positron emitters and gamma emitters can be included into the compounds of the present invention by methods which are usual in the field of organic synthesis. Typically, they will be introduced by using a correspondingly labeled starting material when the desired compound of the present invention is prepared. Illustrative methods of introducing detectable labels are described, for instance, in US 2012/0302755.

The position at which the detectable label is to be attached to the compounds of the present invention is not particularly limited.

### Definitions

Within the meaning of the present application the following definitions apply:

“Alkyl” refers to a saturated straight or branched organic moiety consisting of carbon and hydrogen atoms. Examples of suitable alkyl groups have 1 to 6 carbon atoms, preferably 1 to 4 carbon atoms, and include methyl, ethyl, propyl, isopropyl, n-butyl, t-butyl and isobutyl.

“Alkylene” refers to a divalent saturated, linear or branched organic moiety consisting of carbon and hydrogen atoms. Examples of suitable alkylene groups have 1 to 6 carbon atoms, preferably 1 to 4 carbon atoms, and include methylene, ethylene, propylene, isopropylene, n-butylene, t-butylene and isobutylene.

“5- to 7-Membered monocyclic group, which can optionally contain one or more heteroatoms selected from N and O in addition to the nitrogen atom which is attached to the tricyclic group” refers to a monocyclic organic moiety which contains carbon atoms and at least the nitrogen atom which is attached to the tricyclic group. Optionally one or more further heteroatoms selected from N and O can be present. Examples of the “5- to 7-membered cyclic group, which can optionally contain one or more heteroatoms selected from N and O in addition to the nitrogen atom which is attached to the tricyclic group” include pyrrolidinyl, oxazolidinyl, piperidinyl, morpholinyl, piperazinyl, azepanyl, and diazepanyl.

“7- to 9-Membered bicyclic group, which can optionally contain one or more heteroatoms selected from N and O in addition to the nitrogen atom which is attached to the tricyclic group” refers to a bicyclic organic moiety which contains carbon atoms and at least the nitrogen atom which is attached to the tricyclic group. Optionally one or more further heteroatoms selected from N and O can be present. The term “bicyclic” covers ring systems in which two rings are fused, two attached spiro rings as well as two rings which are attached via a sequence of atoms (bridgehead). Examples of the “7- to 9-membered bicyclic group, which can optionally contain one or more heteroatoms selected from N and O in addition to the nitrogen atom which is attached to the tricyclic group” include azabicyclo[3.1.1]heptyl, azabicyclo[3.2.1]octyl, azaspiro[3.3heptyl, and azaspiro[3.5]nonyl.

“Alkoxy” refers to the group —O-alkyl or —O-alkylene-.

Fluoro refers to [F-19]fluoro and [F-18]fluoro

An “amine protecting group” is any protecting group which is suitable for protecting an amine group during an envisaged chemical reaction. Examples of suitable protecting groups are well-known to a person skilled in the art. Suitable protecting groups are discussed, e.g., in the textbook Greene and Wuts, Protecting groups in Organic Synthesis, third edition, page 494-653, which is included herein by reference. Specific preferred examples of the amine-protecting groups include tert-butyloxycarbonyl (Boc), p-tolylsulfonyl (Tosyl), trimethylsilylethoxycarbonyl (Teoc), carbobenzyloxy (Cbz), p-methoxybenzyl carbonyl (Moz or MeOZ), 9-fluorenylmethyloxycarbonyl (Fmoc), acetyl (Ac), triphenylmethyl (Trt) or trifluoroacetyl.

A “hydroxy protecting group” is any protecting group which is suitable for protecting a hydroxy group during an envisaged chemical reaction. Examples of suitable protecting groups are well-known to a person skilled in the art. Examples thereof include but are not limited to ethers and silyl ethers. Suitable protecting groups are discussed, e.g., in the textbook Greene and Wuts, Protecting groups in Organic Synthesis, third edition, page 23-148, which is included herein by reference. Specific preferred examples of the hydroxy-protecting groups include methoxymethyl (MOM), 2-methoxyethoxymethyl (MEM), 2-(trimethylsilyl)ethoxymethyl (SEM), tetrahydropyranyl (THP), triisopropylsilyl (TIPS), t-butyldimethylsilyl (TBDMS), or t-butyldiphenylsilyl (TBDPS).

A “leaving group” LG is any leaving group which can be attached to a saturated carbon atom and means an atom or group of atoms can be replaced by another atom or group of atoms. Examples are given e.g. in Synthesis (1982), 85-125, table 2 (p. 86; (the last entry of this table 2 needs to be corrected: “n-C₄F₉S(O)₂—O— nonaflat” instead of “n-C₄H₉S(O)₂—O-nonaflat”), Carey and Sundberg, Organische Synthese, (1995), 279-281, table 5.8; or Netscher, Recent Res. Dev. Org. Chem., 2003, 7, 71-83, schemes 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pages 15 to 50, particularly scheme 4 page 25, scheme 5 page 28, table 4 page 30, FIG. 7 page 33). Preferably, the “leaving group” is halogen (e.g., Br, I, Cl), an alkyl sulfonate leaving group, or an aryl sulfonate leaving group.

An “alkyl sulfonate leaving group” includes any group which comprises a —OSO₂—R group, with R being selected from the group consisting of C₁₋₄ alkyl and perfluoro(C₁₋₄)alkyl. This includes but is not limited to methyl sulfonyloxy, trifluoromethyl sulfonyloxy and nonafluorobutyl sulfonyloxy.

An “aryl sulfonate leaving group” includes any group which comprises a —OSO₂—R group, with R being selected from the group consisting of aryl which can be optionally substituted by C₁₋₄ alkyl, halogen and nitro. This includes but is not limited to (4-methylphenyl) sulfonyloxy, (4-bromo-phenyl)sulfonyloxy, (4-nitro-phenyl) sulfonyloxy, (2-nitro-phenyl) sulfonyloxy, (4-isopropyl-phenyl) sulfonyloxy, (2,4,6-tri-isopropyl-phenyl) sulfonyloxy, (2,4,6-trimethyl-phenyl) sulfonyloxy, (4-tert-butyl-phenyl) sulfonyloxy and (4-methoxy-phenyl) sulfonyloxy.

In a more preferred embodiment, “aryl sulfonate leaving group” is methyl sulfonyloxy, (4-methylphenyl) sulfonyloxy, or trifluoromethyl sulfonyloxy.

Compounds of the present invention having one or more optically active carbons can exist as racemates and racemic mixtures, stereoisomers (including diastereomeric mixtures and individual diastereomers, enantiomeric mixtures and single enantiomers, mixtures of conformers and single conformers). The term “stereoisomers and mixtures thereof” is intended to mean racemates and racemic mixtures, stereoisomers (including diastereomeric mixtures and individual diastereomers, enantiomeric mixtures and single enantiomers, mixtures of conformers and single conformers). The mixtures of stereoisomers can be optically active or non-optically active. All isomeric forms are included in the present invention. Also included in this invention are all salt forms, polymorphs, hydrates and solvates.

“Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18th ed., Mack Publishing Company, Easton, Pa., 1990, p. 1445, the disclosure of which is hereby incorporated by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The patients or subjects in the present invention are typically animals, particularly mammals, more particularly humans.

Tau as used herein refers to a highly soluble microtubule binding protein mostly found in neurons and includes the major 6 isoforms, cleaved or truncated forms, and other modified forms such as arising from phosphorylation, glycosylation, glycation, prolyl isomerization, nitration, acetylation, polyamination, ubiquitination, sumoylation and oxidation.

Neurofibrillary Tangles (NFTs) as used herein refer to insoluble aggregates of the hyperphosphorylated tau protein containing paired helical filaments and straight filaments. Their presence is a hallmark of AD and other diseases known as tauopathies.

The preferred definitions given in the “Definition”-section apply to all of the embodiments described below unless stated otherwise.

Diagnostic Procedures

The compounds of the present invention are suitable for imaging of neurodegenerative disorders. The compounds of the present invention are particularly suitable for imaging of tau protein aggregates. With respect to tau protein, the compounds of the present invention are able to bind to pathologically aggregated tau, hyperphosphorylated tau, neurofibrillary tangles, paired helical filaments, straight filaments, neurotoxic soluble oligomers, polymers and fibrils. The compounds of the present invention are particularly suitable for binding to various types of tau aggregates.

Due to the above binding characteristics, the compounds of the present invention are suitable for use in the diagnosis of disorders associated with tau protein aggregates. The compounds of the present invention are particularly suitable for positron emission tomography imaging of tau deposits.

Diseases involving tau aggregates are generally listed as tauopathies and these include, but are not limited to, Alzheimer's disease (AD), familial AD, Creutzfeldt-Jacob disease, dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis, Parkinsonism-dementia complex of Guam, non-Guamanian motor neuron disease with neurofibrillary tangles, argyrophilic grain disease, corticobasal degeneration, diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease, progressive subcortical gliosis, progressive supranuclear palsy (PSP), subacute sclerosing panencephalitis, tangle only dementia, postencephalitic Parkinsonism, myotonic dystrophy, tau panencephalopathy, AD-like with astrocytes, certain prion diseases (GSS with tau), mutations in LRRK2, Hallervorden-Spatz disease, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, and white matter tauopathy with globular glial inclusions, etc (Williams et al., Intern. Med. J., 2006, 36, 652-60).

Due to their design the compounds of the present invention are particularly suitable for use in the diagnosis of Alzheimer's disease, as well as other neurodegenerative tauopathies such as Creutzfeldt-Jacob disease, dementia pugilistica, amyotrophic lateral sclerosis, argyrophilic grain disease, corticobasal degeneration, frontotemporal dementia with Parkinsonism linked to chromosome 17, Pick's disease, progressive supranuclear palsy (PSP), tangle only dementia, Parkinson dementia complex of Guam, Hallervorden-Spatz disease and fronto-temporal lobar degeneration.

In the methods of diagnosing a disorder associated with tau protein aggregates such as Alzheimer's disease, or a predisposition therefor in a subject, the method comprising:
a) administering to the mammal a diagnostically effective amount of a compound of the present invention;
b) allowing the compound of the present invention to distribute into the tissue of interest (such as brain tissue or body fluids such as cerebrospinal fluid (CSF)); and
c) imaging the tissue of interest, wherein an increase in binding of the compound of the present invention to the tissue of interest compared to a normal control level of binding indicates that the subject is suffering from or is at risk of developing a disorder associated with tau protein aggregates.

The compounds of the present invention can be used for imaging of tau protein aggregates in any sample or a specific body part or body area of a patient which suspected to contain a tau protein aggregate. The compounds of the present invention are able to pass the blood-brain barrier. Consequently, they are particularly suitable for imaging of tau protein aggregates in the brain, as well as in body fluids such as cerebrospinal fluid (CSF).

In diagnostic applications, the compound of the present invention is preferably administered in a diagnostic composition comprising the compound of the invention. A “diagnostic composition” is defined in the present invention as a composition comprising compounds of the present invention in a form suitable for administration to mammals such as humans. Preferably a diagnostic composition further comprises a physiologically acceptable carrier, diluent, adjuvant or excipient. Administration is preferably carried out by injection of the composition as an aqueous solution. Such a composition may optionally contain further ingredients such as solvents (including ethanol), buffers; pharmaceutically acceptable solubilisers (e.g., cyclodextrins or surfactants such as Pluronic, Tween or phospholipids); and pharmaceutically acceptable stabilisers or antioxidants (such as ascorbic acid, gentisic acid or para-aminobenzoic acid). The dose of the compound of the present invention will vary depending on the exact compound to be administered, the weight of the patient, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

Diagnosis of a tau disorder or of a predisposition to a tau-associated disorder in a patient may be achieved by detecting the specific binding of a compound according to the invention to the tau protein aggregates in a sample or in situ, which includes:
(a) bringing the sample or a specific body part or body area suspected to contain the tau protein aggregate into contact with a compound of the invention which binds the tau protein aggregate,
(b) allowing the compound of the invention to bind to the tau protein aggregate to form a compound/tau protein aggregate complex (hereinafter “compound/tau protein aggregate complex” will be abbreviated as “compound/protein aggregate complex”),
(c) detecting the formation of the compound/protein complex,
(d) optionally correlating the presence or absence of the compound/protein complex with the presence or absence of tau protein aggregates in the sample or specific body part or area, and
(e) optionally comparing the amount of the compound/protein to a normal control value, wherein an increase in the amount of the compound/protein compared to a normal control value may indicate that the patient is suffering from or is at risk of developing a tau-associated disorder.

After the sample or a specific body part or body area has been brought into contact with the compound of the present invention, the compound is allowed to bind to the tau protein aggregate. The amount of time required for binding will depend on the type of test (e.g., in vitro or in vivo) and can be determined by a person skilled in the field by routine experiments.

The compound which has bound to the tau protein aggregate can be subsequently detected by any appropriate method. A preferred method is positron emission tomography (PET).

The presence or absence of the compound/protein is then optionally correlated with the presence or absence of tau protein aggregates in the sample or specific body part or area. Finally, the amount of the compound/protein can be compared to a normal control value which has been determined in a sample or a specific body part or body area of a healthy subject, wherein an increase in the amount of the compound/protein compared to a normal control value may indicate that the patient is suffering from or is at risk of developing a tau-associated disorder.

The present invention also relates to a method of determining the amount of tau protein aggregate in a tissue and/or a body fluid. This method comprises the steps of:
(a) providing a sample representative of the tissue and/or body fluid under investigation;
(b) testing the sample for the presence of tau protein aggregate with a compound of the present invention;
(c) determining the amount of compound bound to the tau protein aggregate; and
(d) calculating the amount of tau protein aggregate in the tissue and/or body fluid.

The sample can be tested for the presence of tau protein aggregate with a compound of the present invention by bringing the sample into contact with a compound of the invention, allowing the compound of the invention to bind to the tau protein aggregate to form a compound/protein aggregate complex and detecting the formation of the compound/protein complex as explained above.

Monitoring minimal residual disorder in a patient suffering from a disorder associated with tau protein aggregates who has been treated with a medicament with a compound according to the invention may be achieved by:
(a) bringing a sample or a specific body part or body area suspected to contain a tau protein aggregate into contact with a compound of the present invention;
(b) allowing the compound to bind to the tau protein aggregate to form a compound/protein aggregate complex;
(c) detecting the formation of the compound/protein aggregate complex;
(d) optionally correlating the presence or absence of the compound/protein aggregate complex with the presence or absence of tau protein aggregate in the sample or specific body part or body area; and
(e) optionally comparing the amount of the compound/protein aggregate to a normal control value, wherein an increase in the amount of the aggregate compared to a normal control value may indicate that the patient may still suffer from a minimal residual disease.

How steps (a) to (e) can be conducted has already been explained above.

Predicting responsiveness of a patient suffering from a disorder associated with tau protein aggregates and being treated with a medicament can be achieved by
(a) bringing a sample or a specific body part or body area suspected to contain a tau protein aggregate into contact with a compound of the present invention;
(b) allowing the compound to bind to the tau protein aggregate to form a compound/protein aggregate complex;
(c) detecting the formation of the compound/protein aggregate complex;
(d) optionally correlating the presence or absence of the compound/protein aggregate complex with the presence or absence of tau protein aggregate in the sample or specific body part or body area; and
(e) optionally comparing the amount of the compound/protein aggregate to a normal control value.

How steps (a) to (e) can be conducted has already been explained above.

In the method for predicting responsiveness the amount of the compound/protein complex can be optionally compared at various points of time during the treatment, for instance, before and after onset of the treatment or at various points of time after the onset of the treatment. A change, especially a decrease, in the amount of the compound/protein complex may indicate that the patient has a high potential of being responsive to the respective treatment.

A compound according to the present invention can also be incorporated into a test kit for detecting a tau protein aggregate. The test kit typically comprises a container holding one or more compounds according to the present invention and instructions for using the compound for the purpose of binding to a tau protein aggregate to form a compound/protein complex and detecting the formation of the compound/protein complex such that presence or absence of the compound/protein complex correlates with the presence or absence of the tau protein aggregates.

The term “test kit” refers in general to any diagnostic kit known in the art. More specifically, the latter term refers to a diagnostic kit as described in Zrein et al., Clin. Diagn. Lab. Immunol., 1998, 5, 45-49.

Diagnostic Compositions

The compounds of the present invention can be used in diagnosis of neurodegenerative disorders, especially disorders associated with Tau protein aggregates.

The invention also provides a diagnostic composition which comprises an effective amount of a compound of formulae (I) in admixture with a pharmaceutically acceptable carrier, diluent, adjuvant or excipient, suitable for administration to mammals such as humans.

Pharmaceutically acceptable excipients are well known in the pharmaceutical art, and are described, for example, in Remington's Pharmaceutical Sciences, 15^{th} Ed., Mack Publishing Co., New Jersey (1975). The pharmaceutical excipient can be selected with regard to the intended route of administration and standard pharmaceutical practice. The excipient must be acceptable in the sense of being not deleterious to the recipient thereof.

Pharmaceutically useful excipients that may be used in the formulation of the diagnostic composition of the present invention may comprise, for example, carriers, vehicles, diluents, solvents such as monohydric alcohols such as ethanol, isopropanol and polyhydric alcohols such as glycols and edible oils such as soybean oil, coconut oil, olive oil, safflower oil cottonseed oil, oily esters such as ethyl oleate, isopropyl myristate, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-ß-cyclodextrin, polyvinylpyrrolidone, low melting waxes, and ion exchange resins.

If the compounds of the present invention are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds are best used in the form of a sterile aqueous solution which may contain other excipients. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

A proposed dose of the compounds according to the present invention for administration to a human is in the range of 100-600 MBq of a fluorine-18 labeled compound of the present invention, preferably in the range of 150-450 MBq.

The pharmaceutical compositions of the invention can be produced in a manner known per se to the skilled person as described, for example, in Remington's Pharmaceutical Sciences, 15^{th} Ed., Mack Publishing Co., New Jersey (1975).

Diseases that can be detected and monitored with the compounds of the present invention can be associated with the formation of abnormal protein structures, in particular abnormal beta-sheet structures. In the context of the present invention, an abnormal protein structure is a protein structure that arises when a protein or peptide refolds from its natural occurring conformation in healthy individuals, into a beta-sheet three-dimensional structure, which is associated with a pathological condition. Likewise, an abnormal beta-sheet structure in the context of the present invention is a beta-sheet structure that arises when a protein or peptide refolds from its natural occurring conformation in healthy individuals, into a different beta-sheet structure, which is associated with a pathological condition.

In particular, in one embodiment diseases or disorders that can be detected and monitored with the compounds of the present invention are diseases or conditions associated tau proteins aggregates.

The diseases or conditions that can be detected and monitored with the compounds of the present invention include neurodegenerative disorders such as tauopathies. Examples of diseases and conditions which can be detected and monitored are caused by or associated with the formation of neurofibrillary lesions. This is the predominant brain pathology in tauopathy. The diseases and conditions comprise a heterogeneous group of neurodegenerative diseases or conditions including diseases or conditions which show co-existence of tau and amyloid pathologies. Examples of the diseases and conditions which can be treated, alleviated or prevented include Alzheimer's disease (AD), Creutzfeldt-Jacob disease, dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis, Parkinsonism-dementia complex of Guam, non-Guamanian motor neuron disease with neurofibrillary tangles, argyrophilic grain disease, corticobasal degeneration, diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease, progressive subcortical gliosis, progressive supranuclear palsy (PSP), subacute sclerosing panencephalitis, tangle only dementia, postencephalitic Parkinsonism, myotonic dystrophy, tau panencephalopathy, AD-like with astrocytes, certain prion diseases (GSS with tau), mutations in LRRK2, Hallervorden-Spatz disease, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, white matter tauopathy with globular glial inclusions, traumatic stress syndrome, epilepsy, Lewy body dementia (LBD), hereditary cerebral hemorrhage with amyloidosis (Dutch type), mild cognitive impairment (MCI), multiple sclerosis, Parkinson's disease, HIV-related dementia, adult onset diabetes, senile cardiac amyloidosis, endocrine tumors, glaucoma, ocular amyloidosis, primary retinal degeneration, macular degeneration (such as age-related macular degeneration (AMD)), optic nerve drusen, optic neuropathy, optic neuritis, lattice dystrophy. Preferably the diseases and conditions which can be treated, alleviated or prevented include Alzheimer's disease, as well as other neurodegenerative tauopathies such as Alzheimer's disease (AD), Creutzfeldt-Jacob disease, dementia pugilistica, amyotrophic lateral sclerosis, argyrophilic grain disease, corticobasal degeneration, frontotemporal dementia with Parkinsonism linked to chromosome 17, Pick's disease, progressive supranuclear palsy (PSP), tangle only dementia, Parkinson dementia complex of Guam, Hallervorden-Spatz disease and fronto-temporal lobar degeneration.

The compounds of the present invention can be synthesized by one of the general methods shown in the following schemes. These methods are only given for illustrative purposes and should not to be construed as limiting.

General Synthetic Schemes for the Preparation of Tricyclic Building Blocks of this Invention:

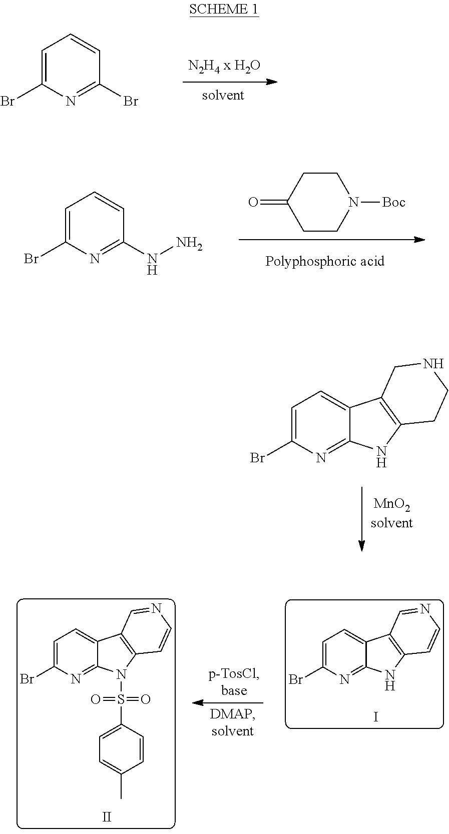

Commercially available 2,6-dibromopyridine was heated under reflux with hydrazine hydrate in a solvent to afford the desired hydrazine-pyridine derivative after purification. The hydrazine-pyridine derivative was then treated with commercially available N-Boc-piperidone under the conditions of a Fischer-indole synthesis using polyphosphoric acid to afford the corresponding azaindole derivative as a free base after purification. Oxidation of the azaindole derivative with manganese dioxide in a solvent afforded the desired tricyclic building block I after purification. Protection of the NH-moiety with a suitable protecting group (tosyl, methane sulfonyl, etc) in a solvent and in the presence of a base and 4-dimethylaminopyridine afforded the desired tricyclic building block II after purification.

General Synthetic Scheme for the Preparation of Compounds of this Invention:

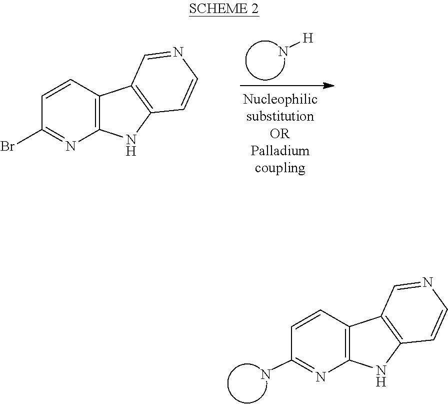

Tricyclic building block I was treated with cycloamine/bicycloamine derivatives containing a fluoro moiety in a solvent using a microwave to enable a nucleophilic substitution reaction to afford the desired final compounds after purification. In an alternative approach, tricyclic building block I was treated with cycloamine/bicycloamine derivatives containing a fluoro moiety in a solvent using a suitable palladium catalyst, ligand and base under Buchwald-Hartwig cross coupling reaction conditions to afford the desired final compounds after purification.

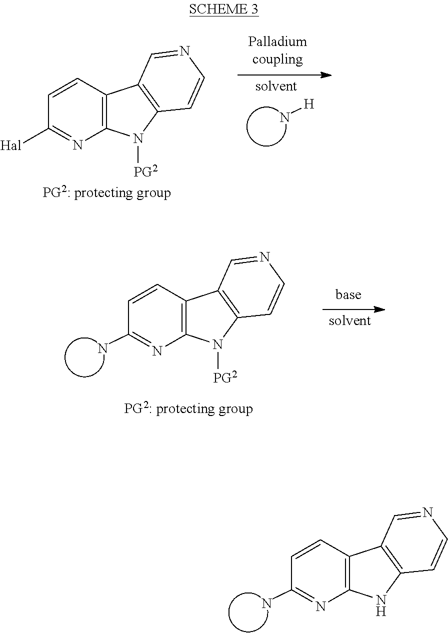

Tricyclic building block II (Hal=Br) was treated cycloamine/bicycloamine/spirocycloamine derivatives containing a fluorine moiety in a solvent via palladium-catalyzed cross coupling (Buchwald-Hartwig amination) conditions to afford the desired coupling products after purification. Cleavage of the protecting group (PG², i.e. tosyl, methane sulfonyl, etc:) in the presence of base in a solvent afforded the desired final compounds after purification.

General Synthetic Scheme for the Preparation of Precursor Compounds of this Invention:

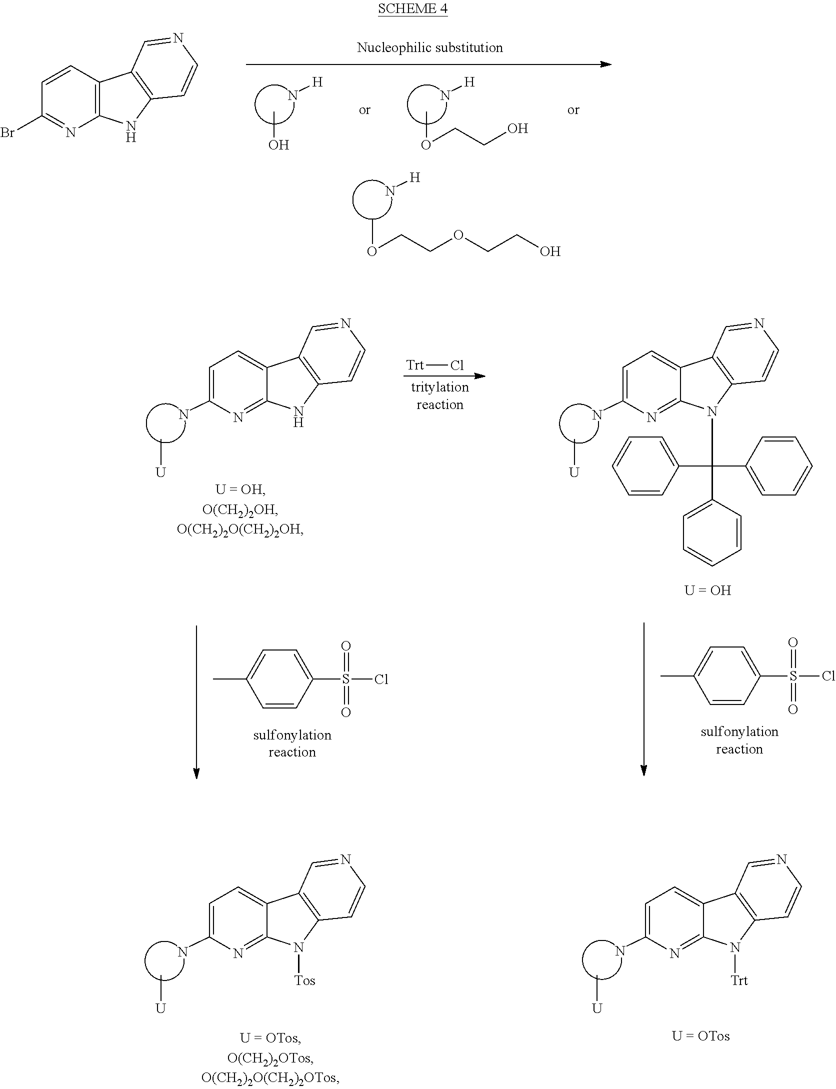

Tricyclic building blocks were treated with cycloamine/bicycloamine derivatives containing a hydroxyl-moiety in a solvent using a microwave to enable a nucleophilic substitution reaction to afford the desired alcohol derivatives after purification. The alcohol derivatives with U═OH, O(CH₂)₂OH and O(CH₂)₂O(CH₂)₂OH were treated with a suitable sulfonylation reagent (CH₃SO₂Cl, p-Tos-Cl) in a solvent to afford the desired precursor compounds to allow the introduction of the ¹⁸F-label in the following step. In an alternative approach, alcohol derivatives with U═OH can be treated with trityl chloride ((C₆H₅)₃C—Cl) to selectively protect the NH-moiety of the tricyclic core and not the secondary alcohol group. The trityl-protected derivatives were treated with a suitable sulfonylation reagent (CH₃SO₂Cl, p-Tos-Cl) in a solvent to afford the desired precursor compounds to allow the introduction of the ¹⁸F-label in the following step.

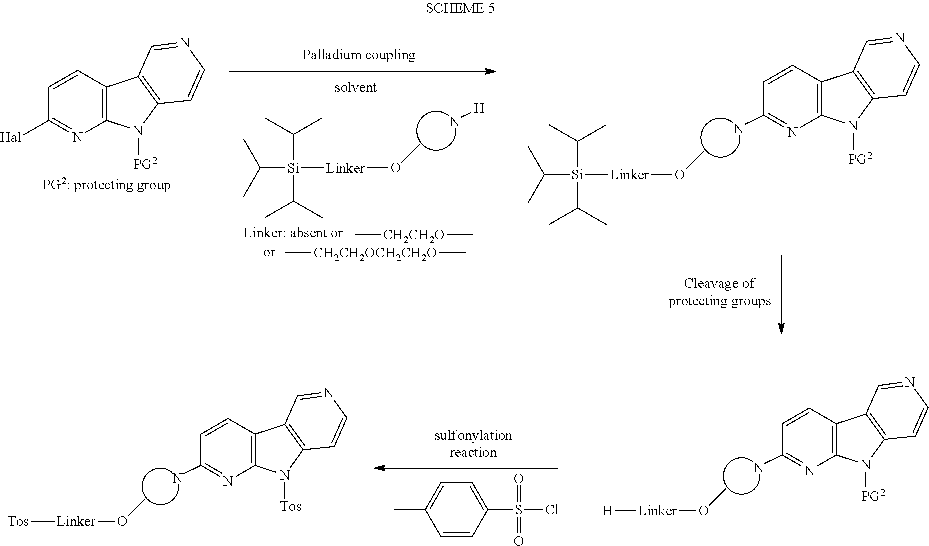

Tricyclic building blocks (Hal=Br, Cl) bearing a protecting group (PG², i.e. tosyl, methane sulfonyl, etc:) were treated with cycloamine/bicycloamine/spirocycloamine derivatives containing a silyl-protected alcohol moiety in the absence or presence of a linker (—CH₂CH₂O—, —CH₂CH₂OCH₂CH₂O—) in a solvent via palladium-catalyzed cross coupling (Buchwald-Hartwig amination) conditions to afford the corresponding coupling products after purification. The silyl group and the PG²-protecting group are cleaved at the same time by treatment with tetrabutyl ammonium fluoride to afford the alcohol derivatives containing a NH-moiety at the pyrrole ring. Sulfonylation of the aliphatic hydroxyl and pyrrole NH-moieties with p-Tos-Cl in a solvent afforded the desired precursor compounds after purification to allow the introduction of the ¹⁸F-label in the following step

General Synthesis of ¹⁸F-Labeled Compounds of the Present Invention

Compounds having the formula (I) which are labeled by ¹⁸F can be prepared by reacting a compound of formula (II) with an ¹⁸F-fluorinating agent, so that the leaving group LG is replaced by ¹⁸F. The preparation optionally includes the cleavage of a protecting group.

Any suitable ¹⁸F-fluorinating agent can be employed. Typical examples include H¹⁸F, alkali or alkaline earth ¹⁸F-fluorides (e.g., K¹⁸F, Rb¹⁸F, Cs¹⁸F, and Na¹⁸F). Optionally, the ¹⁸F-fluorination agent can be used in combination with a chelating agent such as a cryptand (e.g.: 4,7,13,16,21,24-hexaoxa-1,10-diazabicyclo[8.8.8]-hexacosane—Kryptofix®) or a crown ether (e.g.: 18-crown-6). Alternatively, the ¹⁸F-fluorinating agent can be a tetraalkylammonium salt of ¹⁸F or a tetraalkylphosphonium salt of ¹⁸F. Examples thereof include tetrabutylammonium [¹⁸F]fluoride and tetrabutylphosphonium [¹⁸F]fluoride. Preferably, the ¹⁸F-fluorination agent is Cs¹⁸F, K¹⁸F, or tetrabutylammonium [¹⁸F]fluoride.

The reagents, solvents and conditions which can be used for the ¹⁸F-fluorination are well-known to a skilled person in the field (L. Cai, S. Lu, V. Pike, Eur. J. Org. Chem 2008, 2853-2873; J. Fluorine Chem., 27 (1985):177-191; Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). Preferably, the solvents used in the ¹⁸F-fluorination are DMF, DMSO, acetonitrile, DMA, or mixtures thereof, preferably the solvent is acetonitrile, DMSO.

If desired, the compound having the formula (II) can contain an amine protecting group in order to protect the group Z during the ¹⁸F fluorination reaction. This amine protecting group can be subsequently removed. Methods for removing the amine protecting group are known in the art and include, but are not limited to, acid cleavage.

If desired, the compound of formula (I) can be isolated and/or purified further before use. Corresponding procedures are well-known in the art.

The precursor compounds (II) of the present invention can be provided in a kit which is suitable for producing the compounds of the formula (I) by reaction with a ¹⁸F-fluorinating agent. In one embodiment the kit comprises a sealed vial containing a predetermined quantity of the precursor compound (II) of the present invention. For instance, the kit can contain 1.5 to 75 μmol, preferably 7.5 to 50 μmol, more preferably 10 to 30 μmol of a precursor compound (II) of the present invention. Optionally, the kit can contain further components, such as solvents, solid-phase extraction cartridges, components of the ¹⁸F-fluorinating agent, reagent for cleaving protecting groups, solvent or solvent mixtures for purification, solvents and excipients for formulation.

### EXAMPLES

All reagents and solvents were obtained from commercial sources and used without further purification. Proton (¹H) spectra were recorded on a Bruker DRX-400 MHz NMR spectrometer or on a Bruker AV-400 MHz NMR spectrometer in deuterated solvents. Mass spectra (MS) were recorded on an Advion CMS mass spectrometer. Chromatography was performed using silica gel (Fluka: Silica gel 60, 0.063-0.2 mm) and suitable solvents as indicated in the specific examples. Flash purification was conducted with a Biotage Isolera One flash purification system using HP-Sil or KP-NH SNAP cartridges (Biotage) and the solvent gradient indicated in the specific examples. Thin layer chromatography (TLC) was carried out on silica gel plates with UV detection.

Although some of the present examples do not indicate that the respective compounds were detectably labeled, it is understood that corresponding detectably labeled compounds are intended and can be easily prepared, e.g., by using detectably labeled starting materials, such as starting materials containing C(³H)₃, (¹¹C)H₃ or ¹⁸F.

### Preparative Example 1

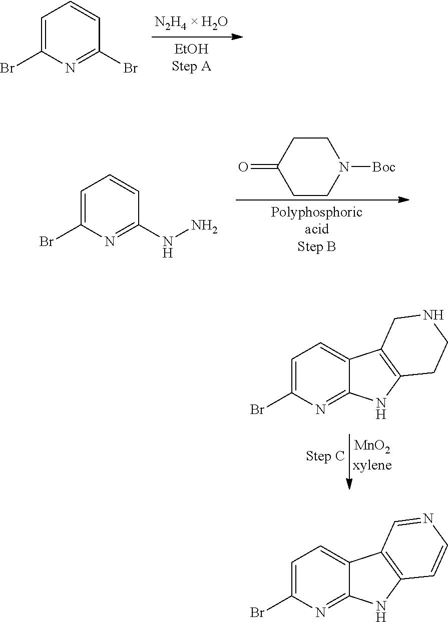 [SMILES: Brc1cccc(Br)n1 ; NNc1cccc(Br)n1 ; CC(C)(C)OC(=O)N1CCC(=O)CC1 ; Brc1ccc2c3c([nH]c2n1)CCNC3 ; Brc1ccc2c(n1)[nH]c1ccncc12]

Step A

Commercially available 2,6-dibromopyridine (4.12 g, 16.6 mmol) was suspended in ethanol (40 mL) and hydrazine hydrate (10 mL, 97.6 mmol) in water (˜50-60%) was added. The mixture was heated in a sand-bath at ˜115° C. for 18 hours. The solvent was removed and the residue was purified by chromatography on silica using ethyl acetate/n-heptane (60/40) to afford the title compound as an off-white solid (3.05 g, 93%).

¹H-NMR (400 MHz, CDCl₃): δ=7.33 (t, 1H), 6.83 (d, 1H), 6.67 (d, 1H), 6.00 (br-s, 1H), 3.33-3.00 (br-s, 2H)

Step B

The title compound from Step A above (10 g, 53.2 mmol) and commercially available 1-Boc-4-piperidone (10.6 g, 53.2 mmol) were added to a 500 mL flask and mixed to become a homogenous blend. Then polyphosphoric acid (80 g, 115% H₃PO₄ basis) was added and the mixture was heated at ˜160° C. in a sand-bath. At ˜120° C. the Boc-protecting group was cleaved resulting in foaming of the reaction mixture. After complete Boc-cleavage the foam collapsed and the dark reaction mixture was stirred at ˜160° C. for 20 hours. The reaction was allowed to cool to room temperature and water (400 mL) was added. The reaction mixture was stirred/sonicated until the gummy material was dissolved. The reaction mixture was then placed in an ice-bath and the pH of the solution was adjusted to pH ˜12 by adding solid sodium hydroxide pellets (exothermic!). The precipitate was collected by filtration and washed with water (400 mL) to remove salts. The precipitate was dissolved in dichloromethane/methanol (9/1; 1500 mL) by sonication and washed with water (2×400 mL) to remove remaining salts and insoluble material. The organic phase was dried over Na₂SO₄, filtered and the solvents removed under reduced pressure. The dark residue was treated with dichloromethane (100 mL), sonicated for 5 minutes and the precipitate collected by filtration.

The precipitate was washed with dichloromethane (40 mL) and air-dried to afford the title compound a beige solid (3.5 g, 26%).

¹H-NMR (400 MHz, DMSO-d₆): δ=11.5 (br-s, 1H), 7.72 (d, 1H), 7.15 (d, 1H), 3.86-3.82 (m, 2H), 3.06-3.00 (m, 2H), 2.71-2.65 (m, 2H)

Step C

The title compound from Step B above (1.75 g, 6.94 mmol) was suspended in xylene (380 mL) and manganese (IV) oxide (6.62 g, 76.9 mmol) was added. The reaction mixture was then heated at ˜160° C. in a sand-bath for 36 hours. The cooled reaction mixture was evaporated under reduced pressure, the residue suspended in dichloromethane/methanol (1/1; 400 mL) and stirred at room temperature for 30 minutes. The reaction mixture was then filtered through paper filters to remove the manganese (IV) oxide and the filter washed with methanol (50 mL). The combined filtrates were evaporated under reduced pressure and the dark residue purified by chromatography on silica (50 g HP-SIL-cartridge) using a Biotage Isolera system employing an ethyl acetate/heptane gradient (5/95-100/0) to remove unpolar impurities followed by dichloromethane/methanol (9/1->4/1) to afford the title compound as dark yellow solid. The total yield from 2 runs was 1.77 g (51%).

¹H-NMR (400 MHz, DMSO-d₆): δ=12.52 (br-s, 1H), 9.42 (s, 1H), 8.61 (d, 1H), 8.53 (d, 1H), 7.56-7.52 (m, 2H)

### Preparative Example 2

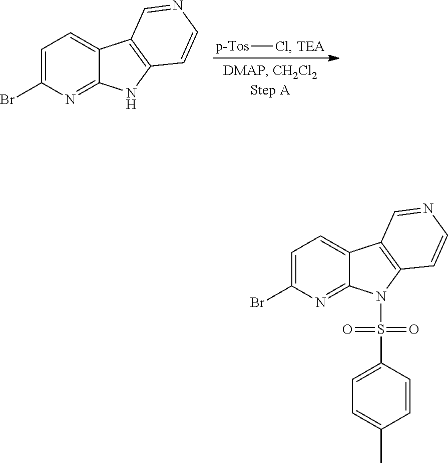

Step A

The title compound from Preparative Example 1 (0.25 g, 1 mmol) was suspended in dichloromethane (15 mL) and triethylamine (1.12 mL, 8.80 mmol) and p-toluene sulfonyl chloride (1.15 g, 6.06 mmol) were added. After the addition of 4-dimethylaminopyridine (0.0245 g, 0.198 mmol), the reaction mixture was stirred at room temperature for 18 hours. To the reaction mixture was then added a 2 M aqueous sodium hydroxide solution (15 mL) and the reaction mixture was stirred at room temperature for 5 minutes. The reaction mixture was diluted with dichloromethane (200 mL) and water (50 mL). The organic phase was separated, washed with water/brine (1/1; 50 mL), dried over Na₂SO₄, filtered and the solvent removed under reduced pressure. The dark residue was purified by chromatography on silica (HP-SIL cartridge) using a Biotage Isolera system employing an ethyl acetate/n-heptane gradient (5/95->100/0->100/0) to afford the title compound as off-white solid (0.324 g, 80%).

¹H-NMR (400 MHz, DMSO-d₆): δ=9.48 (s, 1H), 8.77 (d, 1H), 8.64 (d, 1H), 8.26 (s, 1H), 8.03 (d, 2H), 7.78 (d, 1H), 7.44 (d, 2H), 2.33 (s, 3H)

### Preparative Example 3

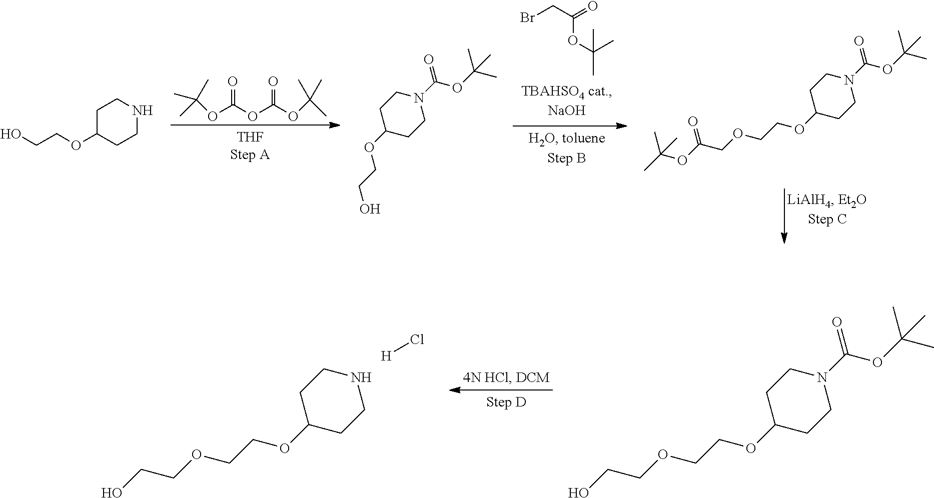

Step A

To a suspension of commercially available 2-(piperidin-4-yloxy)ethanol (0.702 g, 4.83 mmol) in THF (20 mL) was added di-tert-butyl dicarbonate (1.26 g, 5.80 mmol). The reaction mixture was stirred at room temperature for 6 hours. The reaction mixture was concentrated in vacuo to afford the title compound (1.2 g, 100%).

¹H-NMR (400 MHz, DMSO-d₆) δ=4.58-4.50 (m, 1H), 3.62 (m, 3H), 3.45 (m, 4H), 3.09-2.89 (m, 2H), 1.83-1.71 (m, 2H), 1.39 (s, 9H), 1.31 (m, 2H)

Step B

To a solution of the title compound from Step A above (1.2 g, 4.89 mmol), tetra-butyl ammonium hydrogen sulfate (0.083 g, 0.25 mmol) and tert-butyl 2-bromoacetate (1.431 g, 7.34 mmol) in water (1 mL) and toluene (20 mL) was added over a period of 1 hour a solution of NaOH (6.98 g, 175 mmol) in water (10 mL). Then, the reaction mixture was stirred at room temperature for 5 hours. Water was added and the toluene phase was removed under reduced pressure. The aqueous phase was further extracted with toluene. The combined organics were dried over Na₂SO₄, filtered and concentrated in vacuo. The residue was purified on HP-Sil SNAP cartridges using a Biotage Isolera One purification system employing an n-heptane/EtOAc gradient (90/10->60/40) to afford the title compound (1.07 g, 61%).

¹H-NMR (400 MHz, DMSO-d₆) δ=3.99 (s, 2H), 3.67-3.52 (m, 6H), 3.51-3.41 (m, 1H), 3.08-2.92 (m, 2H), 1.83-1.72 (m, 2H), 1.42 (s, 9H), 1.38 (s, 9H), 1.30 (m, 2H)

Step C

To a suspension of lithium aluminum hydride (0.136 g, 3.57 mmol) in diethyl ether (20 mL) at 0° C. was added a solution of the title compound from Step B above (1.07 g, 2.98 mmol) in diethyl ether (10 mL) over 30 min. Then, the reaction mixture was stirred at 0° C. for 2 hours. Then, water (0.13 mL), 15% NaOH (0.13 mL) and water (0.39 mL) were sequentially added at 0° C. to the reaction mixture. The crude product was then allowed to warm to room temperature. After 30 min, the solid was filtered and the mother liquor was concentrated under reduced pressure to afford the title compound (0.882 g, 100%).

¹H-NMR (400 MHz, DMSO-d₆) δ=4.60-4.50 (m, 1H), 3.69-3.56 (m, 2H), 3.56-3.37 (m, 9H), 2.99 (t, 2H), 1.77 (ddt, 2H), 1.39 (s, 9H), 1.30 (dtd, 2H)

Step D

A solution of the title compound from Step C above (0.882 g, 3.05 mmol) in 4 N HCl (5 mL) and dichloromethane (10 mL) was stirred at room temperature for 18 hours. The reaction mixture was concentrated in vacuo to dryness to afford the title compound as a HCl salt (0.680 g, 99%).

¹H-NMR (400 MHz. DMSO-d₆) δ=8.82 (s, 2H), 4.64-4.55 (m, 1H), 3.62-3.45 (m, 7H), 3.45-3.40 (m, 2H), 3.12 (ddd, 2H), 2.99-2.85 (m, 2H), 1.99-1.85 (m, 2H), 1.75-1.60 (m, 2H)

MS (ESI); m/z=190.1 (MH⁺)

### Preparative Example 4

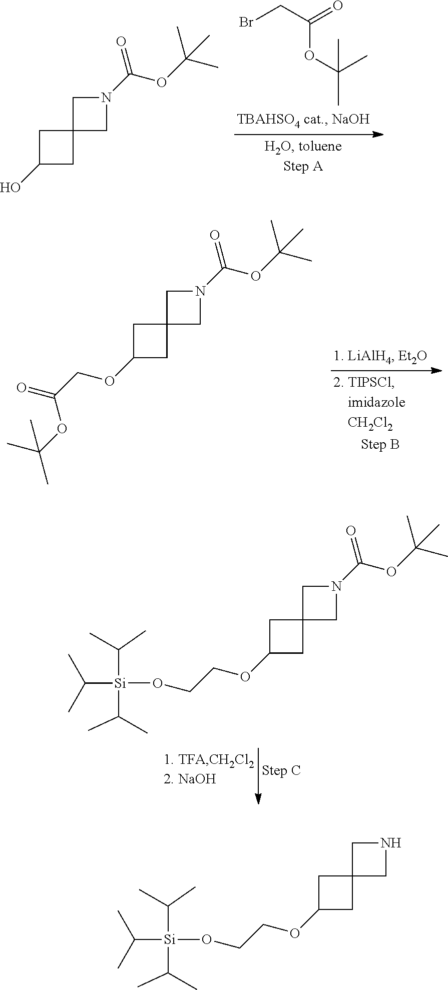

Step A

To a solution of commercially available tert-butyl 6-hydroxy-2-azaspiro[3.3]heptane-2-carboxylate (2.0 g, 9.38 mmol), tetrabutyl ammonium hydrogen sulfate (159 mg, 0.47 mmol) and tert-butyl 2-bromoacetate (2.74 g, 14.07 mmol) in water (1 mL) and toluene (40 mL) were added over 1 hour a solution of NaOH (13.13 g, 328 mmol) in water (20 mL). Then, the reaction mixture was stirred at room temperature for 18 hours. Water was added and the toluene phase was removed. The aqueous phase was further extracted with toluene. The combined organics were dried over Na₂SO₄, filtered and concentrated under reduced pressure. The residue was purified on HP-Sil SNAP cartridges using a Biotage Isolera One purification system employing an ethyl acetate/n-heptane gradient (2/98->50/50) to afford the title compound (2.80 g, 91%).

¹H-NMR (400 MHz, CDCl₃) δ=3.94 (p, 1H), 3.88 (s, 2H), 3.86 (d, 4H), 2.48 (ddd, 2H), 2.18 (ddd, 2H), 1.47 (s, 9H), 1.42 (s, 9H)

Step B

To a suspension of lithium aluminum hydride (0.398 g, 10.50 mmol) in diethyl ether (40 mL) at 0° C. was added a solution of the title compound from Step A above (2.75 g, 8.40 mmol) in diethyl ether (20 mL) over 30 minutes. Then, the reaction mixture was stirred at 0° C. for 2 hours. Then, water (0.4 mL), 15% NaOH (0.4 mL) and water (1.2 mL) were sequentially added at 0° C. to the reaction mixture. The crude product was then allowed to warm to room temperature. After 30 minutes, the solid was filtered and the mother liquor was concentrated under reduced pressure to afford the alcohol. Then, dichloromethane (40 mL) was added and the reaction mixture was cooled to 0° C. Imidazole (1.14 g, 16.80 mmol), followed by triisopropyl silyl chloride (2.67 mL, 12.60 mmol) were added and the reaction mixture was allowed to warm to room temperature. After 2 hours at room temperature, 1N NaOH was added and the aqueous phase was extracted with dichloromethane several times. The combined organics were dried over Na₂SO₄, filtered and dried under reduced pressure. The residue was purified on HP-Sil SNAP cartridges using a Biotage Isolera One purification system employing an ethyl acetate/n-heptane gradient (2/98->30/70) to afford the title compound (2.50 g, 72% over two steps).

¹H-NMR (400 MHz, CDCl₃) 5=3.96-3.83 (m, 5H), 3.79 (t, 2H), 3.42 (t, 2H), 2.53-2.40 (m, 2H), 2.08 (ddd, 2H), 1.42 (s, 9H), 1.16-1.00 (m, 21H)

Step C

To a solution of the title compound from Step B above (2.5 g, 6.04 mmol) in dichloromethane (15 mL) was added at 0° C. trifluoro acetic acid (4.66 mL, 60.4 mmol). Then, the reaction mixture was stirred at room temperature for 4 hours. The reaction mixture was carefully made alkaline to pH 10 using 1 N NaOH and the aqueous phase was extracted several times with dichloromethane. The combined organics were dried over Na₂SO₄, filtered and dried to afford the title compound (1.61 g, 85%).

¹H-NMR (400 MHz. DMSO-d₆) δ=3.81 (q, 1H), 3.71 (t, 2H), 3.45 (s, 2H), 3.40 (s, 2H), 3.32 (t, 2H), 2.37 (ddd, 2H), 1.94-1.83 (m, 2H), 1.11-0.88 (m, 21H)

### Preparative Example 5

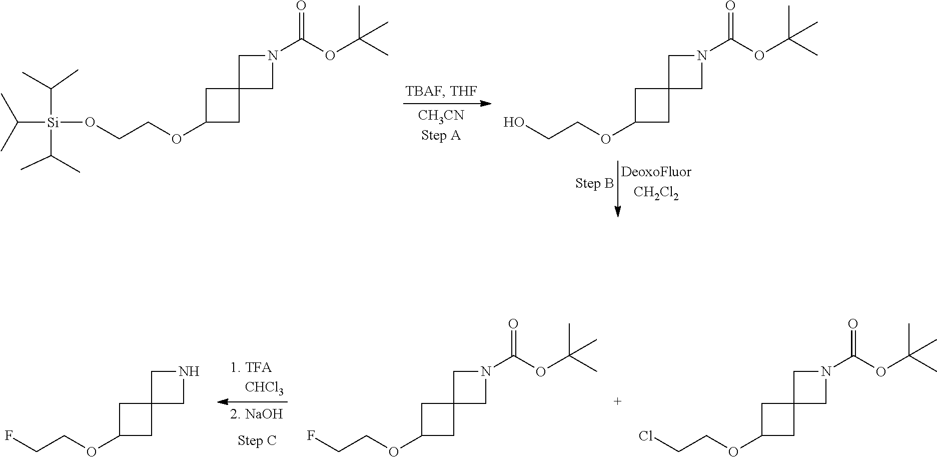 [SMILES: CC(C)[Si](OCCOC1CC2(C1)CN(C(=O)OC(C)(C)C)C2)(C(C)C)C(C)C ; CC(C)(C)OC(=O)N1CC2(CC(OCCO)C2)C1 ; FCCOC1CC2(CNC2)C1 ; CC(C)(C)OC(=O)N1CC2(CC(OCCF)C2)C1 ; CC(C)(C)OC(=O)N1CC2(CC(OCCCl)C2)C1]

Step A

The title compound from Preparative Example 4 Step A (0.336 g, 0.813 mmol) was dissolved in acetonitrile (10 mL) and 1 M solution of tetra-butyl ammonium floride in tetrahydrofuran (1 mL, 1 mmol) was added. The reaction mixture was stirred at room temperature for 30 hours and the solvents removed under reduced pressure. The yellow residue was purified by chromatography on silica (HP-SIL cartridge) using a Biotage Isolera system employing an ethyl acetate/n-heptane gradient (5/95->100/0->100/0) to afford the title compound as colorless oil (0.174 g, 83%).

¹H-NMR (400 MHz, CDCl₃): δ=3.90 (q, 1H), 3.91 (s, 2H), 3.88 (s, 2H), 3.73 (t, 2H), 3.45 (t, 2H), 2.54-2.47 (m, 2H) 2.16-2.09 (m, 2H), 1.45 (s, 9H)

Step B

The title compound from Step A above (0.174 g, 0.637 mmol) was dissolved in dichloromethane (10 mL) and the reaction mixture cooled to −10° C. in an ice-salt bath. At −10° C. a 50% solution of bis(2-methoxyethyl)aminosulfur trifluoride (Deoxo-Fluor) in tetrahydrofuran (0.9 mL, 2.13 mmol) was added and stirring at −10° C. was continued for 1 hour. The reaction mixture was then allowed to warm to room temperature and stirred at room temperature for 1 hour. The reaction mixture was poured into a mixture of saturated ammonium chloride (30 mL) and dichloromethane (100 mL). The organic phase was separated, dried over Na₂SO₄, filtered and the solvent removed under reduced pressure. The residue was purified by chromatography on silica (HP-SIL cartridge) using a Biotage Isolera system employing an ethyl acetate/n-heptane gradient (5/95->30/70->30/70) to afford the less polar chloro-byproduct (0.035 g, 19%, colorless oil) and the more polar title compound as colorless oil (0.077 g, 44%).

Analytical Data Less Polar Chloro-Derivative:

¹H-NMR (400 MHz, CDCl₃): δ=3.90 (q, 1H), 3.90 (2, 2H), 3.87 (s, 2H), 3.60 (s, 4H), 2.54-2.47 (m, 2H), 2.18-2.11 (m, 2H), 1.45 (s, 9H)

MS (ESI); m/z=276.37/278.35 [M+H]⁺

Analytical data more polar title compound:

¹H-NMR (400 MHz, CDCl₃): δ=4.61-4.59 (m, 1H), 4.57-4.55 (m, 1H), 3.92 (q, 1H), 3.90 (s, 2H), 3.88 (s, 2H), 3.64-3.62 (m, 1H), 3.56-3.54 (m, 1H); 2.55-2.49 (m, 2H), 2.18-2.12 (m, 2H), 1.45 (s, 9H)

MS (ESI); m/z=260.41 [M+H]

Step C

The title compound from Step B above (0.077 g, 0.298 mmol) was dissolved in chloroform (5 mL) and treated at 0° C. with trifluoro acetic acid (0.23 mL, 2.98 mmol). The reaction mixture was stirred at 0° C. for 1 hour and then at room temperature for 30 hours. The reaction mixture was diluted with dichloromethane (50 mL) and water (15 mL). The pH was adjusted to pH ˜12 by the addition of a 1M sodium hydroxide solution. The organic phase was separated and the aqueous phase extracted with dichloromethane (30 mL). The combined organic phase was washed with a brine/water mixture (1/1; 20 mL), dried over Na₂SO₄, filtered and the solvent removed under reduced pressure. to afford the title compound as pale orange oil (0.053 g) which was used without further purification.

### Preparative Example 6

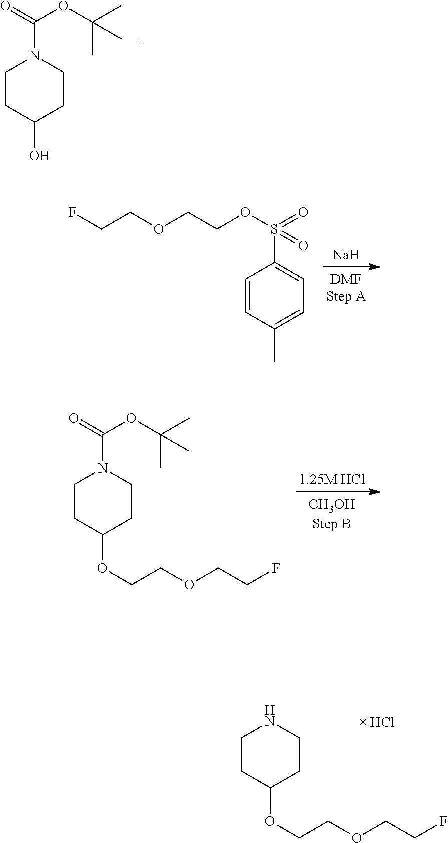

Step A

To a solution of tert-butyl 4-hydroxy-piperidine-1-carboxylate (0. mg, 2.484 mmol) in N,N′-dimethylformamide (20 mL) at 0° C. was added sodium hydride 95% (0.088 m, 3.48 mmol). Then, the reaction was stirred at room temperature for 1 hour. The reaction was cooled to 0° C. again and 2-(2-fluoroethoxy)ethyl 4-methylbenzenesulfonate (1.955 g, 7.45 mmol) was added. After 5 minutes at 0° C., the reaction mixture was allowed to warm to room temperature and stirred for 18 hours. The reaction mixture was then quenched by addition of saturated ammonium chloride solution (10 mL). Ethyl acetate (120 mL) was added and the organic phase was washed with water (3×100 mL), dried over sodium sulfate and concentrated under reduced pressure. The residue was purified by column chromatography using 5 to 25% ethyl acetate in n-heptane to afford the title compound (0.193 g, 27%).

¹H-NMR (400 MHz, DMSO-d₆) δ=4.62-4.40 (m, 2H), 3.76-3.57 (m, 4H), 3.55 (s, 4H), 3.46 (tt, 1H), 3.09-2.92 (m, 2H), 1.85-1.70 (m, 2H), 1.39 (s, 11H)

Step B

A solution of the title compound from Step A above (0.193 g, 0.662 mmol) in 1.25 M solution of hydrogen chloride in methanol (6 mL) was stirred at room temperature for 4 hours. The reaction was concentrated under reduced pressure to afford the title compound (0.154 g, 100%).

¹H-NMR (400 MHz, DMSO-d₆) δ=9.18-8.60 (m, 2H), 4.51 (dt, 2H), 3.73-3.44 (m, 7H), 3.22-3.02 (m, 2H), 3.02-2.82 (m, 2H), 2.06-1.86 (m, 2H), 1.81-1.57 (m, 2H)

MS (ESI); m/z=192.48 [M+H]⁺

### Preparative Example 7

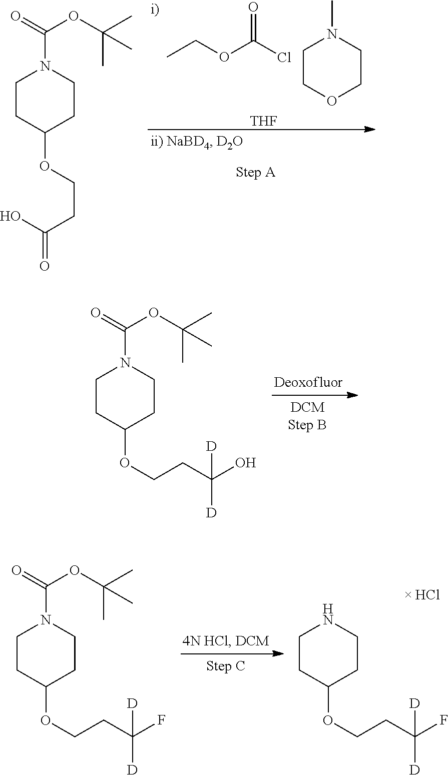

Step A

To a solution of 3-((1-(tert-butoxycarbonyl)piperidin-4-yl)oxy)propanoic acid (0.5345 g, 1.956 mmol) and 4-methylmorpholine (0.236 mL, 2.151 mmol) in tetrahydrofuran (20 mL) at 0° C. was added ethyl carbonochloridate (0.205 mL, 2.151 mmol). After 20 minutes at 0° C., the reaction was filtered and the solid was washed with dry THF (10 mL). The filtrate was cooled to 0° C. and a solution of sodium borodeuteride (106 mg, 2.54 mmol) in 1 mL of deuterium oxide was added slowly. After 30 minutes at 0° C., the reaction was allowed to warm to room temperature and the stirring continued for 20 minutes at room temperature. The crude product was concentrated under reduced pressure to dryness and the solid was dissolved into ethyl acetate (50 mL). The organic phase was washed with brine, dried over sodium sulfate, filtered and dried under reduced pressure. The residue was purified by chromatography on silica (HP-SIL cartridge) using a Biotage Isolera system employing an ethyl acetate/n-heptane gradient (50/50->100/0) to afford the title compound (0.4 g, 78%).

¹H NMR (400 MHz, CDCl₃) 5=3.84-3.69 (m, 2H), 3.66 (t, 2H), 3.56-3.37 (m, 1H), 3.10 (ddd, 2H), 1.92-1.74 (m, 4H), 1.58-1.39 (m, 12H)

MS (ESI); m/z=262.25 (MH⁺)

Step B

To a solution of the title compound from Step A above (0.203 g, 0.777 mmol) in dichloromethane (15 mL) was added deoxofluor (0.573 mL, 1.553 mmol). After 2 hours at room temperature, the reaction mixture was washed with NaOH, brine, dried over sodium sulfate, filtered and dried under reduced pressure. The residue was purified by chromatography on silica (HP-SIL cartridge) using a Biotage Isolera system employing an ethyl acetate/n-heptane gradient (10/90->100/0) to afford the title compound (0.1 g, 49%).

¹H NMR (400 MHz, CDCl₃) δ=3.85-3.64 (m, 2H), 3.64-3.51 (m, 2H), 3.45 (s, 1H), 3.10 (t, 2H), 1.93 (dt, 2H), 1.87-1.72 (m, 2H), 1.54-1.35 (m, 11H)

MS (ESI); m/z=264.27 (MH⁺)

Step C

A solution of the title compound from Step B above (0.1 g, 0.380 mmol) in dichloromethane (5 mL) and 4 M HCl (2 mL) in 1,4-dioxane was stirred at room temperature for 2 hours. The crude product was concentrated under reduced pressure to afford the title compound as an off-white solid (0.076 g, 100%).

¹H NMR (400 MHz, CDCl₃) δ=6 9.48 (s, 2H), 3.65 (t, 1H), 3.54 (t, 2H), 3.31-3.10 (m, 4H), 2.20-2.05 (m, 2H), 2.03-1.92 (m, 3H), 1.89 (t, 1H)

### Preparative Example 8

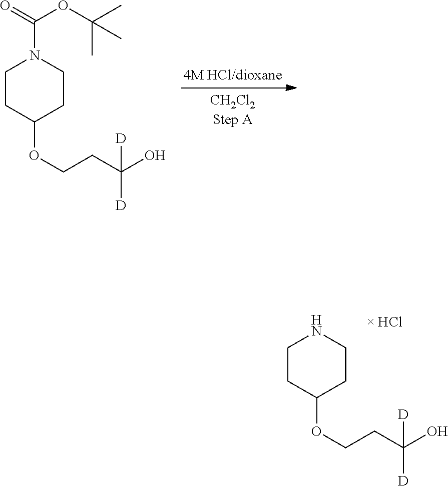 [SMILES: [2H]C([2H])(O)CCOC1CCN(C(=O)OC(C)(C)C)CC1 ; [2H]C([2H])(O)CCOC1CCNCC1]

Step A

A solution of the title compound from Preparative Example 6 Step A (0.19 g, 0.727 mmol) in dichloromethane (2 mL) and 4N HCl (2 mL) in 1,4-dioxane was stirred at room temperature for 2 hours. The crude product was concentrated under reduced pressure the title compound as an off-white solid (0.12 g, 83%).

¹H NMR (400 MHz, CDCl₃) δ=9.47 (s, 2H), 3.67 (s, 1H), 3.59 (t, 2H), 3.34-3.22 (m, 2H), 3.22-3.10 (m, 2H), 2.20-2.06 (m, 2H), 2.06-1.92 (m, 2H), 1.82 (t, 2H), 1.79-1.71 (m, 1H)

MS (ESI); m/z=162.21 (MH⁺)

### Example 1

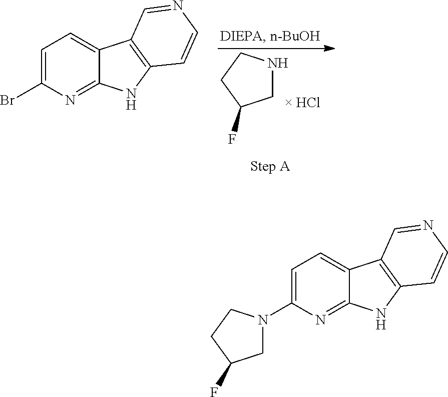

Step A

The title compound from Preparative Example 1 (0.112 g, 0.452 mmol) and commercially available (S)-3-fluoropyrrolidine hydrogen chloride salt (0.168 g, 1.35 mmol) were combined in a 20 mL microwave vial. Then n-butanol (7 mL) was added followed by diisopropyl ethylamine (0.55 mL, 3.15 mmol) and the reaction mixture was heated at 200° C. for 2 hours 30 minutes using a Biotage Initiator microwave. The n-butanol was removed under reduced pressure (25 mbar, 70° C.) and the residue diluted with dichloromethane (100 mL) and water (25 mL). The organic phase was separated, washed with water (25 mL), dried over Na₂SO₄, filtered and the solvents removed under reduced pressure. The dark residue was purified by chromatography on silica (HP-SIL cartridge) using a Biotage Isolera system employing a dichloromethane/methanol gradient (98/2->95/5>90/10) to recover less polar starting material (0.0245 g, 22%, pale yellow solid) and to afford the more polar title compound as pale yellow glass (0.016 g, 14%).

¹H-NMR (400 MHz, DMSO-d₆): b=2.15-2.33 (m, 2H), 3.46-3.68 (m, 2H), 3.72-3.85 (m, 2H), 5.41 (pt, 0.5H), 5.50 (pt, 0.5H), 6.51 (d, 1H), 7.34 (d, 1H), 8.30-8.33 (m, 2H), 9.12 (s, 1H), 12.0 (br-s, 1H)

MS (ESI); m/z=257.20 [M+H]⁺

### Examples 2 to 8 and 17 to 21

Following the coupling procedure as described in Example 1, except using the bromo and amino-alcohol or amino-fluorine derivatives indicated in the table below, the following compounds were prepared. In case the HCl-salt of an amine was used, one additional equivalent of diisopropylethylamine was added.

TABLE 1

| | Amino-alcohol | | |
|---|---|---|---|
| | derivative/ | | 1. Yield |
| | Amino-fluorine | Product | 2. ¹H-NMR |
| Bromo derivative | derivative | Example | 3. MH⁺ (ESI) |
| 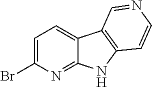 [SMILES: Brc1ccc2c(n1)[nH]c1ccncc12] | 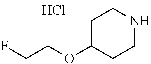 | 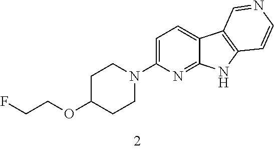 [SMILES: FCCOC1CCN(c2ccc3c(n2)[nH]c2ccncc23)CC1] | 1. 18% 2. ¹H-NMR (400 MHz, DMSO-d₆) δ = 11.80 (br-s, 1H), 9.03 (s, 1H), 8.7-8.24 (m, 2H), 7.25 (d, 1H), 6.82 (d, 1H), 4.61-4.59 (m, 1H), 4.47-4.45 (m, 1H), 4.13-4.06 (m, |
| | | | 2H), 3.75-3.73 (m, |
| | | | 1H), 3.68-3.66 (m, |
| | | | 1H), 3.66-3.61 (m, |
| | | | 1H), 3.27-3.24 (m, |
| | | | 2H), 1.98-1.92 (m, |
| | | | 2H), 1.53-1.48 (m, |
| | | | 2H) |
| | | | 3. 315.40 |
| 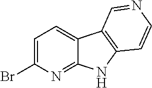 [SMILES: Brc1ccc2c(n1)[nH]c1ccncc12] | 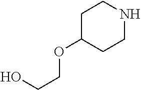 | 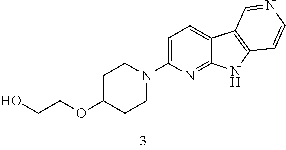 [SMILES: OCCOC1CCN(c2ccc3c(n2)[nH]c2ccncc23)CC1] | 1. 54% 2. ¹H-NMR (400 MHz, DMSO-d₆) δ = 11.72 (br-s, 1H), 9.07 (s, 1H), 8.28 (d, 1H), 8.26 (d, 1H), 7.30 (dd, 1H), 6.83 (d, 1H), 4.58 (t, 1H), 4.12-4.06 (m, 2H), 3.58 (q, 1H), |
| | | | 3.53-3.48 (m, 4H), |
| | | | 3.30-3.24 (m, 2H), |
| | | | 1.96-1.89 (m, 2H), |
| | | | 1.52-1.42 (m, 2H) |
| | | | 3. 312.87 |
| 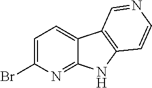 [SMILES: Brc1ccc2c(n1)[nH]c1ccncc12] | 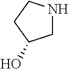 | 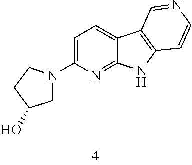 [SMILES: O[C@@H]1CCN(c2ccc3c(n2)[nH]c2ccncc23)C1] | 1. 12% 2. ¹H-NMR (400 MHz, DMSO-d₆) δ = 11.88 (br-s, 1H), 9.06 (s, 1H), 8.27 (d, 1H), 8.25 (d, 1H), 7.30 (d, 1H), 6,44 (d, 1H), 5.00 (d, 1H), 4.46-4.43 (m, 1H), 3.61-3.52 (m, 3H), 3.44-3.40 (m, |
| | | | 1H), 2.11-2.04 (m, |
| | | | 1H), 1.96-1.92 (m, |
| | | | 1H). |
| | | | 3. 255.19 |
| 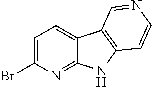 [SMILES: Brc1ccc2c(n1)[nH]c1ccncc12] | 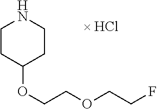 [SMILES: FCCOCCOC1CCNCC1] | 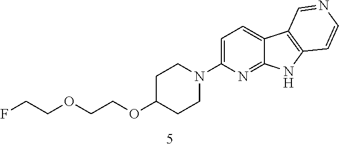 [SMILES: FCCOCCOC1CCN(c2ccc3c(n2)[nH]c2ccncc23)CC1] | 1 25% 2. ¹H-NMR (400 MHz, DMSO-d₆) δ = 11.74 (s, 1H), 9.07 (s, 1H), 8.27 (dd, 2H), 7.29 (d, 1H), 6.82 (d, 1H), 4.52 (dt, 2H) 4.21-3.92 (m, 2H), 3.79-3.49 (m, 7H), 3.30-3.17 (m, |
| | | | 2H), 1.93 (dq, 2H), |
| | | | 1.47 (dtd, 2H). |
| | | | 3. 356.76 |
| 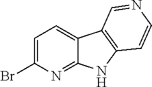 [SMILES: Brc1ccc2c(n1)[nH]c1ccncc12] | 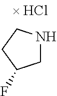 | 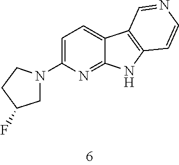 [SMILES: F[C@@H]1CCN(c2ccc3c(n2)[nH]c2ccncc23)C1] | 1. 2% 2. ¹H-NMR (400 MHz, DMSO-d₆) δ = 12.73 (br-s, 1H), 9.30 (s, 1H), 8.42 (dd, 2H), 7.61 (d, 1H), 6.62 (d, 1H), 5.70-5.31 (m, 1H), 3.98-3.68 (m, 2H), 3.57 (m, 2H), 2.40-2.15 (m, 2H). |
| | | | 3. 257.34 |
| 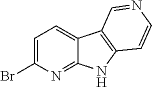 [SMILES: Brc1ccc2c(n1)[nH]c1ccncc12] | 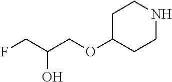 [SMILES: OC(CF)COC1CCNCC1] | 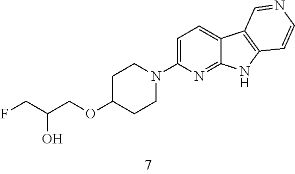 [SMILES: OC(CF)COC1CCN(c2ccc3c(n2)[nH]c2ccncc23)CC1] | 1. 17% 2. ¹H-NMR (400 MHz, DMSO-d₆) δ = 11.74 (s, 1H), 9.08 (s, 1H), 8.48-8.16 (m, 2H), 7.30 (d, 1H), 6.82 (d, 1H), 5.15 (d, 1H), 4.62-4.19 (m, 2H), 4.14-3.92 (m, 2H), 3.92-3.68 (m, 1H), 3.71-3.52 (m, 1H), |
| | | | 3.50-3.42 (m, 2H), |
| | | | 2.01-1.81 (m, 2H), |
| | | | 1.56-1.28 (m, 2H) |
| | | | 3. 345.25 |
| 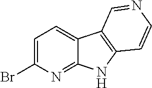 [SMILES: Brc1ccc2c(n1)[nH]c1ccncc12] | 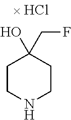 | 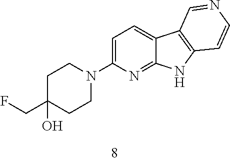 [SMILES: OC1(CF)CCN(c2ccc3c(n2)[nH]c2ccncc23)CC1] | 1. 5% 2. ¹H-NMR (400 MHz, DMSO-d₆) δ = 11.73 (s, 1H), 9.06 (s, 1H), 8.46-8.13 (m, 2H), 7.28 (d, 1H), 6.82 (d, 1H), 4.87 (s, 1H), 4.34- 4.05 (m, 4H), 3.41- 3.33 (m, 2H), 1.67- 1.43 (m, 4H). |
| | | | 3. 301.67 |
| 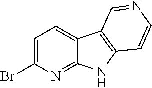 [SMILES: Brc1ccc2c(n1)[nH]c1ccncc12] | 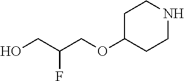 [SMILES: OCC(F)COC1CCNCC1] | 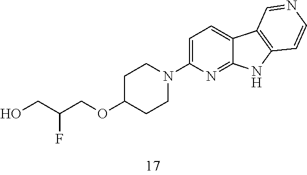 [SMILES: OCC(F)COC1CCN(c2ccc3c(n2)[nH]c2ccncc23)CC1] | 1. 45% 2. ¹H-NMR (400 MHz, DMSO-d₆) δ = 12.10 (br-s, 1H), 9.16 (s, 1H), 8.36 (d, 1H), 8.32 (d, 1H), 7.43 (d, 1H), 6.88 (d, 1H), 4.98- 4.94 (m 1H), 4.66- 4.62 (m, 0.5H), 4.54- 4.50 (m, 0.5H); 4.12- |
| | | | 4.04 (m, 2H), 3.73- |
| | | | 3.55 (m 7H), 1.97- |
| | | | 1.92 (m, 2H), 1.55- |
| | | | 1.45 (m, 2H) |
| | | | 3. 345.52 |
| 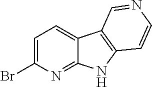 [SMILES: Brc1ccc2c(n1)[nH]c1ccncc12] | 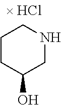 | 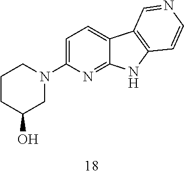 [SMILES: O[C@H]1CCCN(c2ccc3c(n2)[nH]c2ccncc23)C1] | 1. 41% 2. ¹H-NMR (400 MHz, DMSO-d₆) δ = 11.96 (br-s, 1H), 9.13 (s, 1H), 8.33 (d, 1H), 8.28 (d, 1H), 7.39 (d, 1H), 6.82 (d, 1H), 4.92- 4.88 (m, 1H), 4.32- 4.27 (m, 1H), 4.13- 4.08 (m, 1H), 3.55- |
| | | | 3.50 (m, 1H), 3.08- |
| | | | 3.02 (m, 1H); 2.86 |
| | | | (dd, 1H), 1.96-1.92 |
| | | | (m, 1H), 1.80-1.74 (m, |
| | | | 1H), 1.50-1.40 (m, |
| | | | 1H); 1.28-1.22 (m, |
| | | | 1H) |
| | | | 3. 269.03 |
| 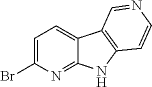 [SMILES: Brc1ccc2c(n1)[nH]c1ccncc12] | 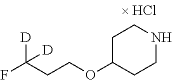 [SMILES: [2H]C([2H])(F)CCOC1CCNCC1] | 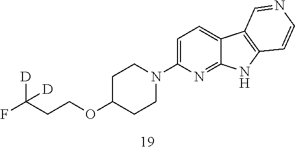 [SMILES: [2H]C([2H])(F)CCOC1CCN(c2ccc3c(n2)[nH]c2ccncc23)CC1] | 1. 18% 2. ¹H-NMR (400 MHz, DMSO-d₆) δ = 11.74 (s, 1H), 9.07 (s, 1H), 8.26 (d, 2H), 7.29 (d, 1H), 6.82 (d, 1H), 4.05 (d, 2H), 3.56 (q, 3H), 3.26 (s, 2H), 2.02- 1.75 (m, 4H), 1.48 (t, |
| | | | 2H) |
| | | | 3. 331.17 |
| 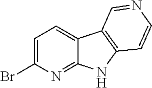 [SMILES: Brc1ccc2c(n1)[nH]c1ccncc12] | 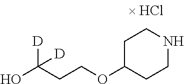 [SMILES: [2H]C([2H])(O)CCOC1CCNCC1] | 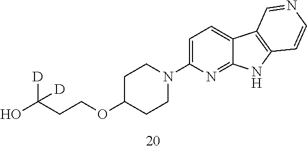 [SMILES: [2H]C([2H])(O)CCOC1CCN(c2ccc3c(n2)[nH]c2ccncc23)CC1] | 1. 50% |
| 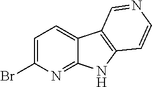 [SMILES: Brc1ccc2c(n1)[nH]c1ccncc12] | 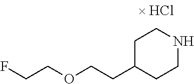 [SMILES: FCCOCCC1CCNCC1] | 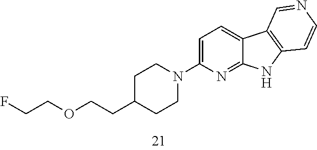 [SMILES: FCCOCCC1CCN(c2ccc3c(n2)[nH]c2ccncc23)CC1] | 1. 44% 2. ¹H-NMR (400 MHz, CDCl₃) δ = 10.01 (s, 1H), 9.13 (s, 1H), 8.46 (d, 1H), 8.14 (d, 1H), 7.29 (d, 1H), 6.69 (d, 1H), 4.69-4.58 (m, 1H), 4.58-4.32 (m, 3H), 3.84-3.68 (m, |
| | | | 1H), 3.60 (d, 2H), 2.98 |
| | | | (td, 2H), 1.92-1.69 |
| | | | (m, 3H), 1.65-1.42 |
| | | | (m, 2H), 1.44-1.18 |
| | | | (m, 2H) |

### Example 9

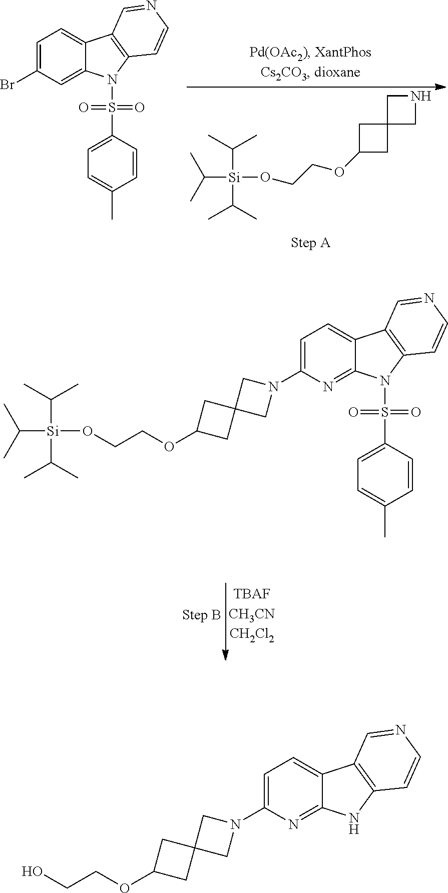 [SMILES: Cc1ccc(S(=O)(=O)n2c3ccncc3c3ccc(Br)cc32)cc1 ; CC(C)[Si](OCCOC1CC2(CNC2)C1)(C(C)C)C(C)C ; Cc1ccc(S(=O)(=O)n2c3ccncc3c3ccc(N4CC5(CC(OCCO[Si](C(C)C)(C(C)C)C(C)C)C5)C4)nc32)cc1 ; OCCOC1CC2(C1)CN(c1ccc3c(n1)[nH]c1ccncc13)C2]

Step A

1,4-Dioxane (12 mL) was degassed for 5 minutes and palladium(II)-acetate (0.018 g, 0.079 mmol) and 5-bis(diphenylphosphino)-9,9-dimethylxanthene (0.138 g, 0.239 mmol) were added. Then the title compound from Preparative Example 2 (0.32 g, 0.795 mmol), the title compound from Preparative Example 4 (0.353 g, 1.128 mmol) and cesium carbonate (1.035 g, 3.18 mmol) were added and heating was continued at ˜120° C. in a sand bath for 2 hours. The reaction mixture was diluted with ethyl acetate (120 mL) and brine/water (1/1; 50 mL), the organic phase separated, dried over Na₂SO₄, filtered and the solvents removed under reduced pressure. The residue was purified by chromatography on silica (HP-SIL cartridges) using a Biotage Isolera system employing an ethyl acetate/n-heptane gradient (5/95->100/0->100/0) to afford the less polar title compound (0.159 g, 31%, off-white solid) and the more polar starting material (0.046 g, 14%) as white solid.

¹H-NMR (400 MHz, CDCl₃) δ=9.02 (s, 1H), 8.55 (d, 1H), 8.30 (d, 1H), 8.10 (d, 2H), 7.96 (d, 1H), 7.29 (d, 2H), 6.24 (d, 1H), 4.16 (s, 2H), 4.12 (s, 2H), 4.06 (q, 1H), 3.86 (t, 2H), 3.52 (t, 2H), 2.65-2.60 (m, 2H), 2.42 (s, 3H), 2.28-2.24 (m, 1H), 1.17-1.07 (m, 21H)

MS (ESI); m/z=636.11 [M+H]⁺

Step B

To a solution of the title compound from Step A above (0.159 g, 0.25 mmol) in acetonitrile (10 mL) and dichloromethane (2 mL) was added a 1 M solution of tetrabutylammonium fluoride in tetrahydrofuran (0.75 mL, 0.75 mmol). The reaction mixture was stirred at room temperature for 5 hours, another batch of tetrabutylammonium fluoride in tetrahydrofuran (0.25 mL, 0.25 mmol) was added and stirring at room temperature was continued for 16 hours. The solvents were evaporated under reduced pressure and the residue was purified by chromatography on silica (HP-SIL cartridges) using a Biotage Isolera system employing dichloromethane/methanol gradient (98/2->90/10->80/20->50/50) to afford the title compound (0.55 g) contaminated with tetrabutylammonium salt.

MS (ESI); m/z=325.56 [M+H]⁺

### Examples 10 to 11

Following the palladium coupling procedure as described in Example 9 Step A, except using the bromo derivatives and amino-fluorine derivatives indicated in the table below, the following compounds were prepared. In case the HCl-salt of an amine was used, one additional equivalent of cesium carbonate was added.

TABLE 2

| | Amino- | | 1. Yield |
|---|---|---|---|
| | fluorine | Product | 2. ¹H-NMR |
| Bromo derivative | derivative | Example | 3. MH⁺ (ESI) |
| 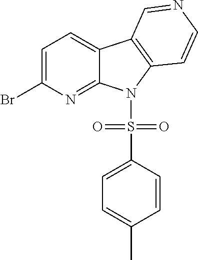 [SMILES: Cc1ccc(S(=O)(=O)n2c3ccncc3c3ccc(Br)nc32)cc1] | 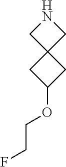 [SMILES: FCCOC1CC2(CNC2)C1] | 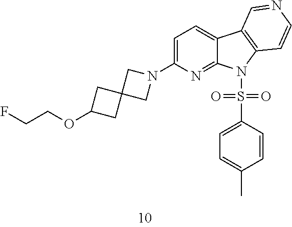 [SMILES: Cc1ccc(S(=O)(=O)n2c3ccncc3c3ccc(N4CC5(CC(OCCF)C5)C4)nc32)cc1] | 1.13% 2. ¹H-NMR (400 MHz, CDCl₃) δ = 9.05 (s, 1H), 8.56 (d, 1H), 8.28 (d, 1H), 8.18 (d, 2H), 7.97 (d, 1H), 7.30 (d, 2H), 6.24 (d, 1H), 4.66- 4.84 (m, 1H), 4.54- 4.52 (m, 1H), 4.17 (s, 2H), 4.14 (s, 2H), 4.07 (q, 1H), 3.72- 3.70 (m, 1H), 3.63- 3.61 (m 1H), 2.68- 2.63 (m, 2H), 2.42 (s, 3H), 2.35-2.28 |
| | | | (m, 2H) |
| 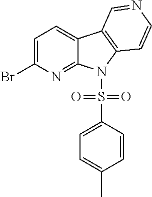 [SMILES: Cc1ccc(S(=O)(=O)n2c3ccncc3c3ccc(Br)nc32)cc1] | 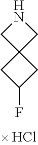 | 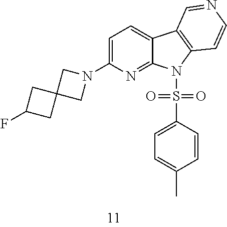 [SMILES: Cc1ccc(S(=O)(=O)n2c3ccncc3c3ccc(N4CC5(CC(F)C5)C4)nc32)cc1] | 1.67% 2. ¹H-NMR (400 MHz, CDCl₃) δ = 9.02 (s, 1H), 8.56 (d, 1H), 8.28 (d, 1H), 8.10 (d, 2H), 7.96 (d, 1H), 7.30 (d, 2H), 6.25 (d, 1H), 5.14 (q, 0.5H), 5.00 (q, 0.5H), 4.18 (s, 2H), 4.16 (s, 2H), 2.77- 2.72 (m, 2H), 2.58- 2.48 (m, 2H), 2.42 |
| | | | (s, 3H) |

### Example 12

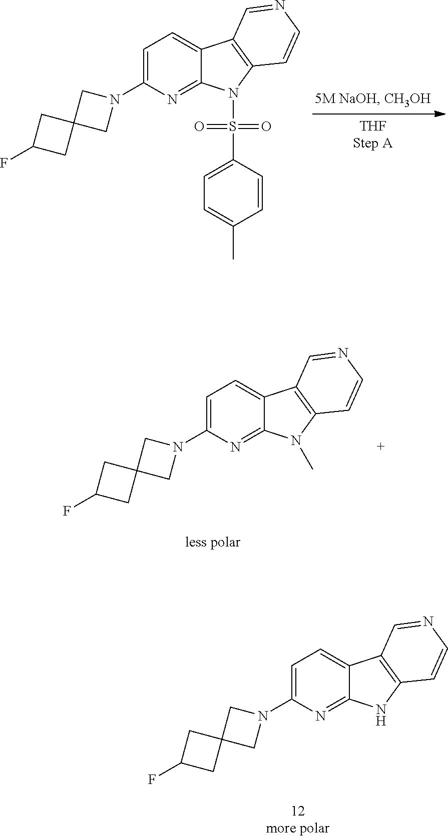 [SMILES: Cc1ccc(S(=O)(=O)n2c3ccncc3c3ccc(N4CC5(CC(F)C5)C4)nc32)cc1 ; Cn1c2ccncc2c2ccc(N3CC4(CC(F)C4)C3)nc21 ; FC1CC2(C1)CN(c1ccc3c(n1)[nH]c1ccncc13)C2]

Step A

The title compound from Example 10 (0.074 g, 0.169 mmol) was dissolved in tetrahydrofuran (5.5 mL) and a 5 M solution of sodium hydroxide in methanol (0.14 mL) was added. The reaction mixture was stirred at room temperature for 4 hours, diluted with water (25 mL) and extracted with dichloromethane (3×50 mL). The organic phase was separated, dried over Na₂SO₄, filtered and the solvents removed under reduced pressure. The residue was purified by preparative TLC using dichloromethane/methanol (9/1) as mobile phase. The bands with the less polar N-methyl derivative and the more polar NH-derivative were cut out and the compounds eluted with dichloromethane/methanol (9/1) to afford the less polar N-methyl derivative (0.0052 g, 10%, beige solid) and the more polar NH-derivative as beige solid (0.0165 g, 35%).

Less polar N-methyl derivative:

¹H-NMR (400 MHz, DMSO-d₆) δ=9.12 (br-s, 1H), 8.40 (br-s, 1H), 8.30 (d, 1H), 7.56 (d, 1H), 6.33 (d, 1H), 5.12 (q, 0.5H), 4.98 (q, 0.5H), 4.10 (s, 2H), 3.98 (s, 2H), 3.80 (s, 3H), 2.72-2.65 (m, 2H), 2.49-2.39 (m, 2H)

MS (ESI); m/z=297.35 [M+H]⁺

More polar NH-derivative title compound (12):

¹H-NMR (400 MHz, DMSO-d₆) δ=11.88 (br-s, 1H), 9.10 (s, 1H), 8.31-8.25 (m, 2H), 7.29 (d, 1H), 6.30 (d, 1H), 5.12 (q, 0.5H), 4.98 (q, 0.5H), 4.06 (s, 2H), 4.04 (s, 2H), 2.71-2.65 (m, 2H), 2.49-2.39 (m, 2H)

MS (ESI); m/z=283.34 [M+H]⁺

### Example 13

Following the N-tosylate cleavage procedure as described in Example 12, except using the N-tosyl derivatives indicated in the table below, the following compounds were prepared.

TABLE 3

| | | 1. Yield |
|---|---|---|
| | Product | 2. ¹H-NMR |
| N-tosyl derivative | Example | 3. MH⁺ (ESI) |
| 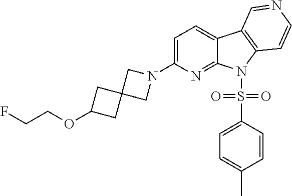 [SMILES: Cc1ccc(S(=O)(=O)n2c3ccncc3c3ccc(N4CC5(CC(OCCF)C5)C4)nc32)cc1] | 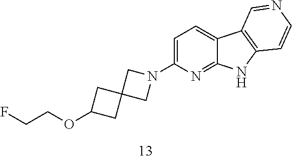 [SMILES: FCCOC1CC2(C1)CN(c1ccc3c(n1)[nH]c1ccncc13)C2] | 1. 41% 2. ¹H-NMR (400 MHz, DMSO-d₆) δ = 11.85 (br-s, 1H), 9.04 (br-s; 1H), 8.25-8.21 (m, 2H), 7.25 (br.s, 1H), 6.26- 6.24 (m, 1H), 4.57-4.55 (m, 1H), 4.45-4.43 (m, 1H), 4.13 (s, 2H), 4.11 (s, 2H), 3.58-3.56 (m, 1H), 2.17-2.10 (m, 2H) ¹H-NMR (400 MHz, |
| | | CD₃OD) δ = 9.01 (br-s, |
| | | 1H), 8.31-8.26 (m, 2H), |
| | | 7.50-7.53 (br-s, 1H), |
| | | 6.35 (d, 1H), 4.46-4.44 |
| | | (m, 1H), 4.12 (s, 2H), |
| | | 4.10 (s, 2H), 3.77-3.75 |
| | | (m, 1H), 3.61-3.59 (m, |
| | | 1H), 2.65-2.58 (m, 2H), |
| | | 2.27-2.20 (m, 2H) |
| | | 3. 327.37 |

### Example 14

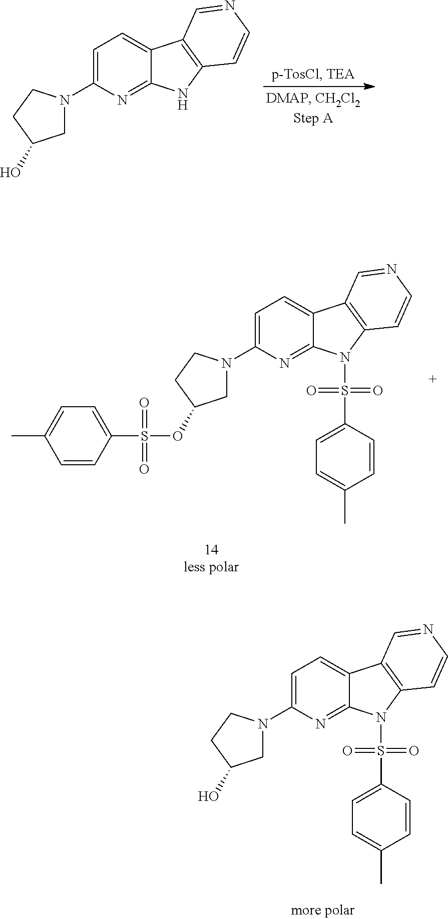 [SMILES: O[C@@H]1CCN(c2ccc3c(n2)[nH]c2ccncc23)C1 ; Cc1ccc(S(=O)(=O)O[C@@H]2CCN(c3ccc4c5cnccc5n(S(=O)(=O)c5ccc(C)cc5)c4n3)C2)cc1 ; Cc1ccc(S(=O)(=O)n2c3ccncc3c3ccc(N4CC[C@@H](O)C4)nc32)cc1]

Step A

The title compound from Example 4 (0.016 g, 0.063 mmol) was suspended in dichloromethane (4 mL) and triethylamine (0.07 mL, 0.5 mmol) and p-toluene sulfonic acid chloride (0.073 g, 0.38 mmol) added. After the addition of 4-dimethylaminopyridine (0.0015 g, 0.012 mmol), the reaction mixture was stirred at room temperature for 18 hours to become a clear, dark solution. Then a 2 M aqueous solution of sodium hydroxide (4 mL) was added and the reaction mixture was stirred vigorously for 3 minutes. The reaction mixture was diluted with dichloromethane (40 mL) and water (10 mL). The organic layer was separated, washed with sat. NaCl/water (1/1; 20 mL), dried over Na₂SO₄, filtered and the solvents removed under reduced pressure. The dark residue was purified by chromatography on silica (HP-SIL cartridge) using a Biotage Isolera system employing an ethyl acetate/n-heptane gradient (5/95->100/0>100/0) to afford the less polar title compound (0.014 g, 39%, off-white solid) and the N-tosyl byproduct as off-white solid (0.0095 g, 37%).

Analytical data title compound (14):

¹H-NMR (400 MHz, DMSO-d₆): δ=9.18 (s, 1H), 8.54 (d, 1H), 8.29 (d, 1H), 8.15 (d, 1H), 7.98 (d, 2H), 7.88 (d, 2H), 7.51 (d, 2H), 7.40 (d, 2H); 6.56 (d, 1H), 5.31-5.30 (m, 1H), 3.72-3.70 (m, 2H), 3.67-3.61 (m, 1H), 3.51-3.45 (m, 1H), 2.43 (s, 3H), 2.34 (s, 3H), 2.25-2.32 (m, 1H); 2.13-2.20 (m, 1H)

MS (ESI); m/z=563.09 [M+H]⁺

Analytical data N-tosyl derivative:

¹H-NMR (400 MHz, DMSO-d₆): δ=9.15 (s, 1H), 8.52 (d, 1H), 8.52 (d, 1H), 8.25 (d, 1H); 8.15 (d, 1H); 8.15 (d, 1H); 8.03 (d, 2H); 7.42 (d, 2H); 6.55 (d, 1H), 5.06 (d, 1H), 4.47-4.45 (m, 1H), 3.63-3.40 (m, 4H), 2.35 (s, 3H), 2.05-2.12 (m, 1H), 1.95-2.01 (m, 1H),

MS (ESI); m/z=409.11 [M+H]⁺

### Examples 15 to 16 and 22 to 23

Following the alkylation procedure as described in Example 14, except using the alcohol derivatives and alkylation reagents indicated in the table below, the following compounds were prepared.

TABLE 4

| | Alkyl- | | 1. Yield |
|---|---|---|---|
| | ation | Product | 2. ¹H-NMR |
| Alcohol derivative | reagent | Example | 3. MH⁺ (ESI) |
| 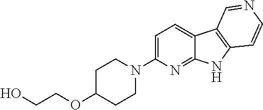 [SMILES: OCCOC1CCN(c2ccc3c(n2)[nH]c2ccncc23)CC1] | 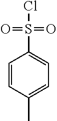 | 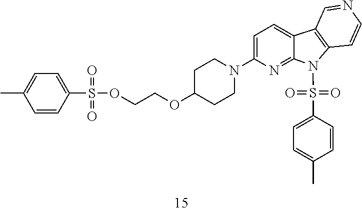 [SMILES: Cc1ccc(S(=O)(=O)OCCOC2CCN(c3ccc4c5cnccc5n(S(=O)(=O)c5ccc(C)cc5)c4n3)CC2)cc1] | 1. 79% 2. ¹H-NMR (400 MHz, DMSO-d₆) δ = 9.18 (s, 1H), 8.55 (d, 1H), 8.27 (d, 1H), 8.16 (d, 1H), 7.96 (d, 2H), 7.80 (d, 2H), 7.45 (d, 2H), 7.42 (d, 2H), 6.95 (d, 1H), 4.20-4.18 (m, 1H), 4.00-3.94 (m, 2H), 3.67-3.65 (m, 2H), 3.55 (q, 1H), 3.32- 3.29 (m, 2H), 2.36 |
| | | | (s, 3H), 2.34 (s, 3H), |
| | | | 1.85-1.77 (m, 2H), |
| | | | 1.38-1.28 (m, 2H) |
| | | | 3. 620.42 |
| 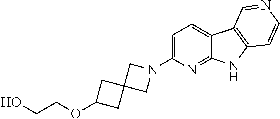 [SMILES: OCCOC1CC2(C1)CN(c1ccc3c(n1)[nH]c1ccncc13)C2] | 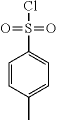 | 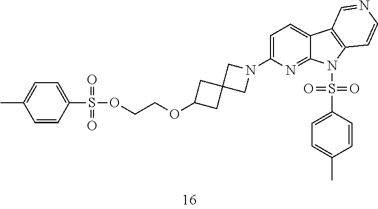 [SMILES: Cc1ccc(S(=O)(=O)OCCOC2CC3(C2)CN(c2ccc4c5cnccc5n(S(=O)(=O)c5ccc(C)cc5)c4n2)C3)cc1] | 1. 12.6% 2. ¹H-NMR (400 MHz, DMSO-d₆) δ = 9.17 (s, 1H), 8.53 (d, 1H), 8.27 (d, 1H), 8.16 (d, 1H), 8.04 (d, 2H), 7.83 (d, 2H), 7.56 (d, 2H), 7.45 (d, 2H), 6.40 (d, 1H), 4.15-4.13 (m, 2H), 4.07-3.98 (m, 5H), 3.88 (q, 1H), 3.49- 3.47 (m, 2H), 2.60- 2.55 (m, 2H), 2.45 |
| | | | (s, 3H), 2.37 (s, 3H), |
| | | | 2.07-1.97 (m, 2H), |
| | | | 3. 633.87 |
| 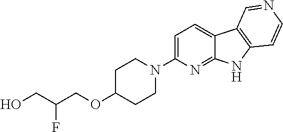 [SMILES: OCC(F)COC1CCN(c2ccc3c(n2)[nH]c2ccncc23)CC1] | 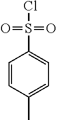 | 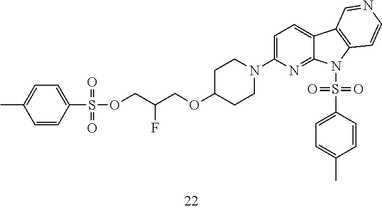 [SMILES: Cc1ccc(S(=O)(=O)OCC(F)COC2CCN(c3ccc4c5cnccc5n(S(=O)(=O)c5ccc(C)cc5)c4n3)CC2)cc1] | 1. 31% 2. ¹H NMR (400 MHz, CDCl₃) δ = 9.03 (s, 1H), 8.57 (d, 1H), 8.38 (d, 1H), 8.04-7.98 (m, 3H), 7.84 (d, 2H), 7.48 (d, 2H), 7.26 (d, 2H), 6.67 (d, 1H), 4.90- 4.86 (m, 0.5H), 4.77- 4.73 (m, 0.5H), 4.34- 4.32 (m, 1H), 4.28- 4.26 (m, 1H), 4.07- 4.00 (m, 2H), 3.78 |
| | | | (d, 1H), 3.74 (d, 1H), |
| | | | 3.67-3.62 (m, 1H), |
| | | | 3.50-3.44 (m, 2H), |
| | | | 2.47 (s, 3H), 2.40 (s, |
| | | | 3H), 1.98-1.93 (m, |
| | | | 2H), 1.68-1.61 (m, 2H) |
| | | | 3. 653.80 |
| 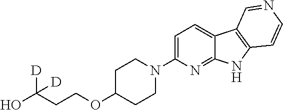 [SMILES: [2H]C([2H])(O)CCOC1CCN(c2ccc3c(n2)[nH]c2ccncc23)CC1] | 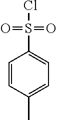 | 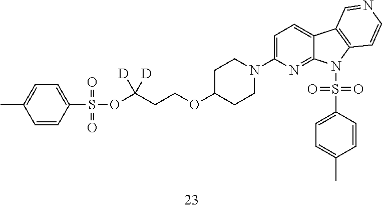 [SMILES: [2H]C([2H])(CCOC1CCN(c2ccc3c4cnccc4n(S(=O)(=O)c4ccc(C)cc4)c3n2)CC1)OS(=O)(=O)c1ccc(C)cc1] | 1. 42% 2. ¹H NMR (400 MHz, CDCl₃) δ = 8.99 (s, 1H), 8.55 (d, 1H), 8.24 (d, 1H), 7.97 (t, 3H), 7.81 (d, 2H), 7.35 (d, 2H), 7.25-7.17 (m, 2H), 6.65 (d, 1H), 4.08- 3.93 (m, 2H), 3.62- 3.47 (m, 3H), 3.47- 3.34 (m, 2H), 2.42 (s, 3H), 2.36 (s, 3H), 1.99-1.82 (m, 4H), |
| | | | 1.36-1.19 (m, 2H) |
| | | | 3. 637.06 |

### Example 24

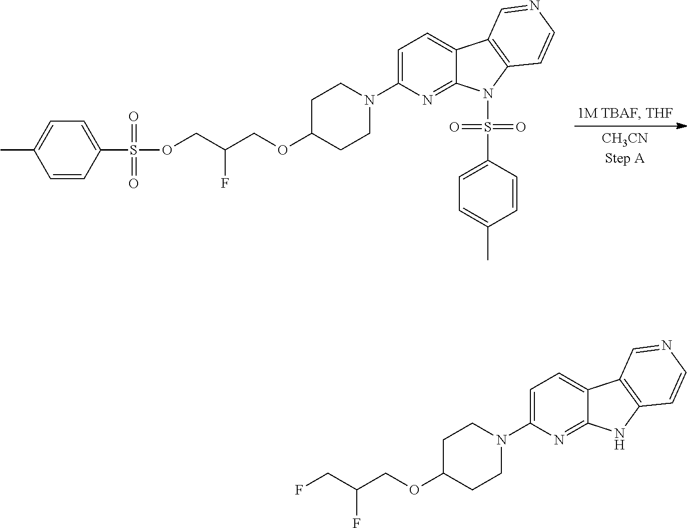 [SMILES: Cc1ccc(S(=O)(=O)OCC(F)COC2CCN(c3ccc4c5cnccc5n(S(=O)(=O)c5ccc(C)cc5)c4n3)CC2)cc1 ; FCC(F)COC1CCN(c2ccc3c(n2)[nH]c2ccncc23)CC1]

Step A

The title compound from Example 22 (0.036 g, 0.055 mmol) was dissolved in acetonitrile (2 mL) and 1 M solution of tetra-butylammoniumfluoride in tetrahydrofuran (0.55 mL, 0.55 mmol) was added. The reaction mixture was heated at 80° C. using a Biotage Initiator microwave. The precipitate was removed by filtration and the filtrate was concentrated using a rotavap. The residue was purified by chromatography on silica (25 g BGB-column) using a Biotage Isolera system employing a dichloromethane/methanol gradient (98/2->90/10->80/20->50/50). Fractions containing the title compound were collected and the solvents were removed in vacuo. In order to remove tetra-butylammonium salts, the residue was purified by chromatography using a Biotage NH-column (11 g) on a Biotage Isolera system employing an ethyl acetate/n-heptane gradient (5/95->100/0) to afford the title compound as a colorless wax (0.0035 g, 18%).

¹H-NMR (400 MHz, DMSO-d₆): δ=11.85 (br-s, 1H), 9.11 (s, 1H), 8.33-8.27 (m, 2H), 7.33 (d, 1H), 6.85 (d, 1H), 5.02-4.92 (m, 0.5H), 4.88-4.79 (m, 0.5H), 4.77-4.53 (m, 2H), 4.08-4.02 (m, 2H), 3.77-3.74 (m, 1H), 3.72-3.68 (m, 1H), 3.67-3.62 (m, 1H), 3.57 (s, 2H), 1.96-1.92 (m, 2H), 1.56-1.46 (m, 2H)

MS (ESI); m/z=347.45 [M+H]⁺

### Example 25

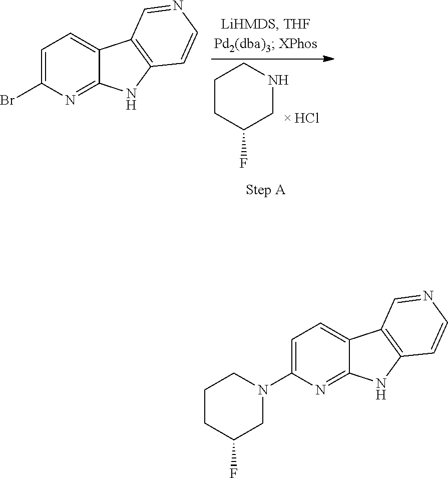

Step A

The title compound from Preparative Example 1 (0.1 g, 0.403 mmol), commercially available (R)-3-fluoropiperidine hydrochloride (0.071 g, 0.52 mmol), tris(dibenzylideneacetone)-dipalladium(0) (0.029 g, 0.036 mmol) and 2-dicyclohexylphosphino-2′,4′,6′-triisopropylbiphenyl (0.031 g, 0.064 mmol) were combined in a microwave vial and a 1 M solution of lithium-bis(trimethylsilyl)amide in tetrahydrofuran (2.5 mL, 2.5 mmol) was added. The reaction mixture was heated at 90° C. in a sand-bath for 1 hour. The reaction mixture was diluted with ethyl acetate (50 mL) and water (20 mL), the organic phase was separated, dried over Na₂SO₄, filtered and the solvents were evaporated in vacuo. The residue was purified by chromatography on silica (25 g HP-Sil-column) using a Biotage Isolera system employing a dichloromethane/methanol gradient (98/2->90/10->80/20). Fractions containing the title compound were collected and the solvents evaporated in vacuo. The residue was again purified by chromatography on silica (25 g BGB-column) using a Biotage Isolera system employing a dichloromethane/methanol gradient (98/2->90/10->80/20) to afford the title compound as an off-white glass (0.025 g, 22%).

¹H-NMR (400 MHz, DMSO-d₆): δ=9.13 (s, 1H), 8.70-8.65 (br-s, 1H), 8.46 (d, 1H), 8.15 (d, 1H), 7.33 (d, 1H), 6.70 (d, 1H), 4.87-4.83 (m, 0.5H), 4.75-4.71 (m, 0.5H), 4.03-3.93 (m, 1H), 3.88-3.80 (m, 1H), 3.70-3.75 (m, 2H), 2.10-1.95 (m, 3H), 1.73-1.68 (m, 1H)

MS (ESI); m/z=271.04 [M+H]⁺

### Examples 26 to 28

Following the alkylation procedure as described in Example 25, except using the bromo- and fluoro-derivatives indicated in the table below, the following compounds were prepared.

TABLE 5

| | | | 1. Yield |
|---|---|---|---|
| | Fluoro | Product | 2. ¹H-NMR |
| Bromo derivative | derivative | Example | 3. MH⁺ (ESI) |
| 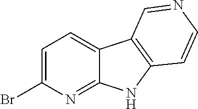 [SMILES: Brc1ccc2c(n1)[nH]c1ccncc12] | 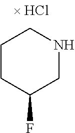 | 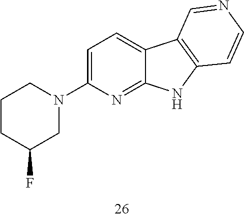 [SMILES: F[C@H]1CCCN(c2ccc3c(n2)[nH]c2ccncc23)C1] | 1. 6% 2. ¹H NMR (400 MHz, CDCl₃) δ = 9.11 (s, 1H), 8.45 (d, 1H), 8.14 (d, 1H), 7.35 (d, 1H), 6.71 (d, 1H), 4.79 (ddt, 1H), 3.93 (m, 2H), 3.67 (m, 2H), 2.01 (m, 3H), 1.68 (dd, 1H), 3. 271.24 |
| 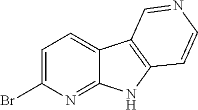 [SMILES: Brc1ccc2c(n1)[nH]c1ccncc12] | 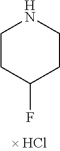 | 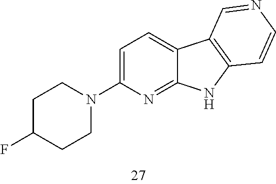 [SMILES: FC1CCN(c2ccc3c(n2)[nH]c2ccncc23)CC1] | 1. 38% 2. ¹H-NMR (400 MHz, DMSO-d₆) δ = 11.78 (s, 1H), 9.09 (s, 1H), 8.30 (dd, 2H), 7.31 (d, 1H), 6.86 (d, 1H), 4.93 (dtt, 1H), 3.86 (ddt, 2H), 3.61 (ddd, 2H), 1.97 (dddt, 2H), 1.75 (ddtd, 2H), 3. 271.23 |
| 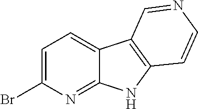 [SMILES: Brc1ccc2c(n1)[nH]c1ccncc12] | 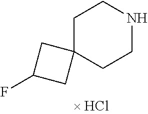 | 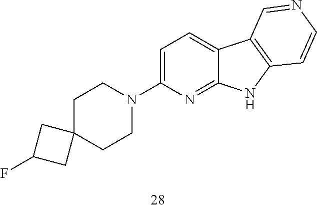 [SMILES: FC1CC2(CCN(c3ccc4c(n3)[nH]c3ccncc34)CC2)C1] | 1. 26% 2. ¹H-NMR (400 MHz, DMSO-d₆) δ = 12.00 (s, 1H), 9.37 (s, 1H), 9.13 (s, 1H), 8.33 (dd, 2H), 7.77 (t, 1H), 7.37 (d, 1H), 7.30 7.16 (m,2H), 6.79 (d, 1H), 6.61- 6.49 (m, 1H), 4.79 (dt, 2H), 4.27 (dt, 2H), 4.10 (q, 1H), 3.18 (d, 3H), |
| | | | 3. 311.39 |

### Example 29

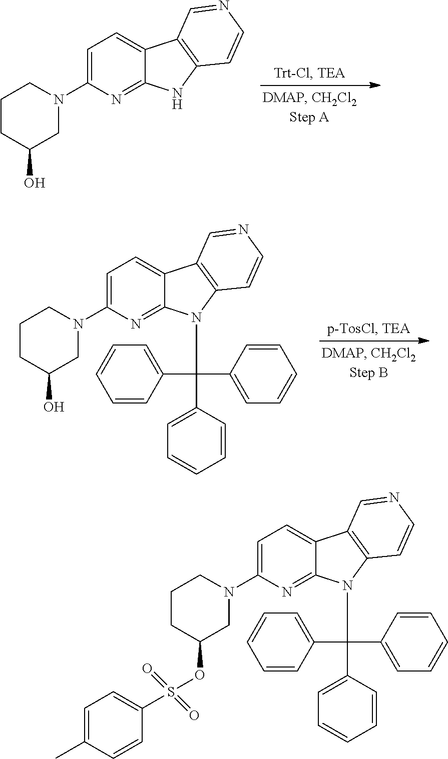 [SMILES: O[C@H]1CCCN(c2ccc3c(n2)[nH]c2ccncc23)C1 ; O[C@H]1CCCN(c2ccc3c4cnccc4n(C(c4ccccc4)(c4ccccc4)c4ccccc4)c3n2)C1 ; Cc1ccc(S(=O)(=O)O[C@H]2CCCN(c3ccc4c5cnccc5n(C(c5ccccc5)(c5ccccc5)c5ccccc5)c4n3)C2)cc1]

Step A

The title compound from Example 18 (0.09 g, 0.33 mmol) was suspended in dichloromethane (5 mL) and triethylamine (0.13 mL, 0.92 mmol) and trityl chloride (0.184 g, 0.66 mmol) was added. After the addition of 4-(dimethylamino)-pyridine (0.079 g, 0.065 mmol), the reaction mixture was stirred at room temperature for 18 hours. The reaction mixture was diluted with dichloromethane (50 mL) and water (25 mL). The organic phase was separated, dried over Na₂SO₄, filtered and the solvents were evaporated in vacuo. The residue was purified by chromatography on silica (25 g BGB-column) using a Biotage Isolera system employing an ethyl acetate/n-heptane gradient (5/95->1000/10->100/0) to afford the title compound as an off-white solid (0.066 g, 39%).

MS (ESI); m/z=511.17 [M+H]⁺

The title compound from Step A above (0.066 g, 0.131 mmol) was dissolved in dichloromethane (3 mL) and triethylamine (0.11 mL, 0.786 mmol) and p-toluene sulfonic acid chloride (0.050 g, 0.262 mmol) were added. After the addition of 4-dimethylaminopyridine (0.003 g, 0.0126 mmol), the reaction mixture was stirred at room temperature for 8 hours. As the reaction was not completed, another batch of triethylamine (0.06 mL) and p-toluene sulfonic acid chloride (0.025 g) was added and stirring at room temperature was continued for 12 hours. Then a 2 M aqueous solution of sodium hydroxide (3 mL) was added and the reaction mixture was stirred vigorously for 3 minutes. The reaction mixture was diluted with dichloromethane (30 mL) and water (15 mL). The organic layer was separated, washed with saturated NaCl/water (1/1; 20 mL), dried over Na₂SO₄, filtered and the solvents were removed under reduced pressure. The dark residue was purified by chromatography on silica (BGB-column) using a Biotage Isolera system employing an ethyl acetate/n-heptane gradient (5/95->100/0>100/0) to afford the title compound (0.055 g, 63%) as an off-white glass/foam.

¹H-NMR (400 MHz, CDCl₃): δ=9.04 (s, 1H), 8.09 (d, 1H), 8.00 (d, 1H), 7.80 (d, 2H), 7.53-7.43 (m, 6H), 7.32 (d, 2H), 7.27-7.17 (m, 10H); 6.38 (d, 1H), 6.32 (d, 1H), 4.46-4.40 (m, 1H), 4.17-4.11 (m, 1H), 3.62-3.56 (m, 1H), 3.25-3.20 (m, 1H); 3.13-3.06 (m, 1H); 2.78-2.70 (m, 1H), 2.45 (s, 3H), 1.85-1.80 (m, 1H), 1.72-1.62 (m, 1H), 1.50-1.50 (m, 1H)

MS (ESI); m/z=665.01 [M+H]⁺

Synthesis of ¹⁸F-Labeled Compounds

General Method (Direct ¹⁸F-Labeling Plus Deprotection)

The tracers were synthesized starting from n.c.a. [¹⁸F]fluoride (1-10 GBq) by a ¹⁸F-direct fluorination. The aqueous [¹⁸F]fluoride solution was trapped on a Sep-Pak Accell Plus QMA light cartridge (Waters) and eluted with a solution K₂CO₃/Kryptofix® 2.2.2. The water was removed using a stream of N₂ at 120° C. and co-evaporated to dryness with MeCN (3×1 mL). Afterwards, the respective dissolved precursor was added to the dried K[¹⁸F]F—K₂₂₂ complex. The reaction vial was sealed and heated for 15 min at 120-160° C. (heating block). For deprotection hydrochloric acid was added and stirred for another 10 min at 110° C. After neutralization using sodium hydroxide solution the reaction mixture was quenched with ammonium formate buffer and trapped on a C-18 Plus cartridge (Waters). The cartridge was washed with water (5 mL), eluted with acetonitrile and subsequently, the crude product was purified via semi-preparative HPLC. The isolated tracer was diluted with water (25 mL), trapped on a C-18 Plus cartridge (Waters), washed with water (5 mL), eluted with ethanol (1 mL).

Representative Examples of Radiolabeling:

### Example ¹⁸F-2

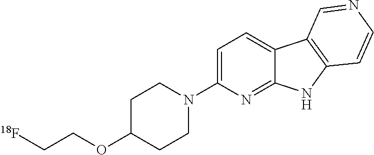 [SMILES: [18F]CCOC1CCN(c2ccc3c(n2)[nH]c2ccncc23)CC1]

¹⁸F-2 (488 MBq) was synthesized according to General Method using precursor molecule from Example 15 (3.5 mg, 5.6 μmol) in acetonitrile (0.5 mL).

The radiochemical purity of 99% was determined by analytical reversed-phase HPLC (t_{R}(RAD-trace)=2.25 min). The identity of ¹⁸F-2 was confirmed by comparing the retention time with the non-radioactive reference from Example 2.

### Example ¹⁸F-1

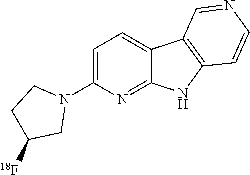 [SMILES: [18F][C@H]1CCN(c2ccc3c(n2)[nH]c2ccncc23)C1]

¹⁸F-1 (168 MBq) was synthesized according to General Method using precursor molecule from Example 14 (2.5 mg, 4.4 μmol) in acetonitrile (0.7 mL).

The radiochemical purity of 100% was determined by analytical reversed-phase HPLC (t_{R}(RAD-trace)=3.2 min). The identity of ¹⁸F-1 was confirmed by comparing the retention time with the non-radioactive reference from Example 1.

Biological Assay Description

Assay 1 (Fluorescence Based Assay):

Direct Binding of Compounds of this Invention to Human Alzheimer's Disease Brain Sections

Frozen sections with a thickness of 20 μM from the amygdala of donors diagnosed with Braak stage V-VI Alzheimer's Disease (Braak, H.; Braak, E. Acta Neuropathol., 1991, 82, 239-259), were purchased from a commercial provider (Tissue Solutions) and kept at −80° C. until use.

Direct Staining of Human Alzheimer's Disease Brain Sections

Brain sections were encircled with pap pen liquid blocker to reduce the volume of solution for the different incubations. Sections were fixed for 15 min at 4° C. with 4% paraformaldehyde and washed three times 5 minutes with PBS at room temperature. Test compounds were incubated on the sections at 100 μM in 50% ethanol in water for 20 min at room temperature in the dark and then washed three times 5 minutes with PBS. To reduce the auto-fluorescence of the tissue, the sections were incubated in a solution of 0.1% Sudan Black (Sigma 199664) in 70% ethanol for 15 min at room temperature in the dark, washed four times for 5 minutes with PBS, and mounted using ProLong Gold Antifade reagent (Invitrogen P36930). Sections were then analyzed on the Nikon Eclipse Ti microscope to detect staining and imaged using Nikon DS-Fi2 camera and NIS-Element AR4.13.1 software.

The title compound of Example 6 shows weak direct staining of tau tangles on AD brain sections at 100 μM.

Tau Radio-Binding Competition Assay

20 μg of human Alzheimer disease brain homogenate (stage Braack V) was incubated with a dilution series of each test compound (1000 to 0.06 nM) in the presence of 800 Bq of [¹⁸F]-labeled Tau-Reference binder. The samples were shaken at 110 rpm for 45 min at 37° C. Samples were then filtered through GF/B 96 well filter plates and two times washed with 300 μl assay buffer (PBS containing 0.1% BSA and 2% DMSO). Thereafter, filter plates were sealed and a Fuji Film Imaging Plate (BAS-SR2025) were placed on top. The imaging plate was analyzed the next day using a Fuji Film BAS-5000. Non-specific signal was determined with samples containing [¹⁸F]-labeled Tau-Reference binder in the presence of assay buffer missing brain substrate and competitor. Specific binding was calculated by subtracting the non-specific signal from the measured sample signal. The unblocked [¹⁸F]-labeled Tau-Reference binder signal was defined as total binding. IC₅₀ values were calculated by Prism V6 (GraphPad) using total binding as 100%.

Monoamine Oxidase A (MAO-A) Assay

Compounds were tested for their capacity to modulate MAO-A activity within the MAO-Glo™ Assay (Promega, Cat.# V1402). In each well of a 96-well plate, 12.5 μl of 4×MAO Substrate solution (end-concentration in assay: 40 μM) were mixed with 12.5 μl of a 4× solution of the corresponding test compound (end-concentrations ranged from 10 μM to 1.37 nM). In the absence of a test compound (i.e. to gauge the total MAO-A activity), the corresponding volume of MAO Reaction Buffer (100 mM HEPES (pH 7.5), 5% glycerol) was added. To initiate the MAO reaction, 25 μl of 2×MAO-A enzyme solution (MAO-A human-recombinant, Sigma-Aldrich, Cat.# M7316; 1 μg MAO-A enzyme/25 μl MAO Reaction Buffer) were added per well and mixed briefly. For negative control reactions, 25 μl of MAO Reaction Buffer were added. Thereafter the plate was incubated at room temperature for 1 hour, following which, 50 μl of reconstituted Luciferin Detection Reagent were added per well and mixed briefly. The plate was then incubated at room temperature for 20 minutes to generate and stabilize the luminescent signal. The luminescent signal was subsequently measured and recorded on the TopCountNXT™ luminescence counter (PerkinElmer). Data were analyzed and graphs were prepared using the Graphpad Prism 6 software.

MAO-B Assay

Compounds were tested for their capacity to modulate MAO-B activity within the MAO-Glo™ Assay (Promega, Cat.# V1402). In each well of a 96-well plate, 12.5 μl of 4×MAO Substrate solution (end-concentration in assay: 4 μM) were mixed with 12.5 μl of a 4× solution of the corresponding test compound (end-concentrations ranged from 10 μM to 1.37 nM). In the absence of a test compound (i.e. to gauge the total MAO-B activity), the corresponding volume of MAO-B Reaction Buffer (100 mM HEPES (pH 7.5), 5% glycerol, 10% dimethyl sulphoxide) was added. To initiate the MAO reaction, 25 μl of 2×MAO-B enzyme solution (MAO-B human—recombinant, Sigma-Aldrich, Cat.# M7441; 1 μg MAO-B enzyme/25 μl MAO-B Reaction Buffer) were added per well and mixed briefly. For negative control reactions, 25 μl of MAO-B Reaction Buffer were added. Thereafter the plate was incubated at room temperature for 1 hour, following which, 50 μl of reconstituted Luciferin Detection Reagent were added per well and mixed briefly. The plate was then incubated at room temperature for 20 minutes to generate and stabilize the luminescent signal. The luminescent signal was subsequently measured and recorded on the TopCountNXT™ luminescence counter (PerkinElmer). Data were analyzed and graphs were prepared using the Graphpad Prism 6 software.

Autoradiography Using Human Brain Tissue

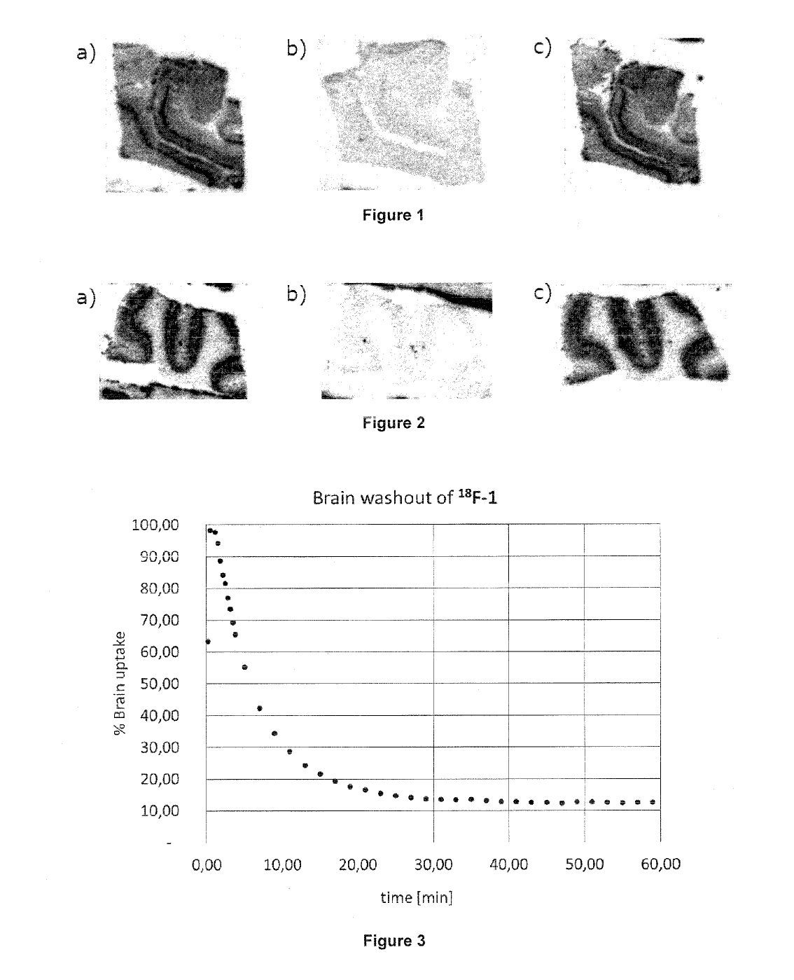

18 micron thick frozen human brain slices and 6 micron thick human FFPE (Formalin-fixed paraffin-embedded) brain slices from the frontal and/or temporal lobes of AD patients were examined via autoradiography. Brain sections were equilibrated for at least 5 min in 1×PBS solution prior to use in the experiment. Each brain section was covered with a solution of the ¹⁸F labeled tracer (200 Bq/μl, 500 μl) in 1×PBS. For blocking experiments with either the corresponding ¹⁸F-labeled compound (termed self-block) or other ¹⁸F-labeled compounds (termed cross-block), an excess of the blocking compound was mixed with the ¹⁸F-compound to yield an end concentration of 10 μM of the ¹⁸F-labeled compound. The brain sections were incubated in the tracer solution at room temperature for 1 h in a humidity chamber, drained thereafter and placed in a slide holder. The slides were then washed sequentially with 1×PBS for 1 min; 70% EtOH in 1×PBS for 2 min; 30% EtOH in 1×PBS for 1 min; and 1×PBS for 1 min. The slides were allowed to air-dry before being placed under Fuji imaging plates in imaging boxes for overnight exposure. The imaging plates were scanned using Fuji BAS-5000 software and the resulting images were analyzed and the signal was quantified using AIDA software. Representative examples of autoradiography studies with ¹⁸F-2 and ¹⁸F-1 are shown in FIG. 1 and FIG. 2.

Strong accumulation of activity was observed in gray matter areas. The specificity of the signal was confirmed by blocking with an excess of the corresponding non-radioactive compound. Additionally, selectivity was demonstrated by cross-blocking with a tracer binding to amyloid-beta. No competition of selected compounds from this invention by amyloid-beta-binding compounds was found.

Brain PK Studies in Mice Using PET Imaging

NMRI mice (weight range 25-35 g) were injected intravenously with the 18F-labeled compounds. Up to 150 μL of 1×PBS, NaCL solution with 10%-15% EtOH or dilution medium (57% water for injections, 18% polyethylene glycol 400, 15% ethanol, 10% water) containing the 18F-labeled compound (2-10 MBq) were injected. Anesthesia with isoflurane and oxygen was induced before injection of the tracer and maintained during the image acquisition period. PET scans were performed using a SIEMENS INVEON small animal PET/CT scanner (Siemens, Knoxville, Tenn.). PET acquisition was started immediately before the radioactive dose was injected into the animal through the tail vein. Images were generated as dynamic scans for 60 minutes. The peak uptake in the brain was set to 100% and washout curves were generated to evaluate the clearance of the activity from the normal brain. Brain wash-out curves for selected compound ¹⁸F-1 is illustrated in FIG. 3.

TABLE 6
Tau and MAO A/B binding data

| | Tau binding | MAO A binding | MAO B binding |
|---|---|---|---|
| Examples | (nM) | (nM) | (nM) |
| 1 | 6 | >1000 | >1000 |
| 2 | 4 | >6000 | >1000 |
| 5 | 6 | >2000 | >1000 |
| 6 | 7 | 940 | >1000 |
| 7 | 3 | 790 | >1000 |
| 8 | 40 | 940 | >1000 |
| 12 | 13 | 570 | >1000 |
| 13 | 16 | >2000 | >1000 |
| 19 | 2.3 | n.d. | n.d. |
| 21 | 1.5 | n.d. | n.d. |
| 24 | 8 | n.d. | n.d. |
| 25 | 5.4 | 1017 | n.d. |
| 26 | 16 | n.d. | n.d. |
| 27 | 3 | n.d. | n.d. |
| 28 | 5.2 | n.d. | n.d. |

n.d.: not determined;

1. A compound of formula (I): 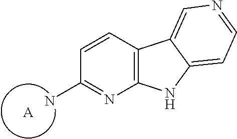
and all stereoisomers and mixtures thereof, pharmaceutically acceptable salts, hydrates, solvates and polymorphs thereof;
wherein 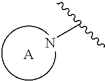
is selected from a 5- to 7-membered monocyclic group and a 7- to 9-membered bicyclic group, which can optionally contain a heteroatom selected from N and O in addition to the nitrogen atom which is attached to the tricyclic group and wherein 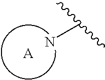
is substituted by a substituent R and is optionally substituted by a substituent R*;
wherein
R is selected from F—, alkylene which is substituted by F, (alkylene-oxy)ₙ- which is substituted by F, (alkylene-oxy)ₙ-alkylene-which is substituted by F, and alkylene-oxy which is substituted by F and OH;
R* is selected from hydroxy, methoxy, methyl and hydroxymethyl;
n is selected from 1, 2 and 3; and
F is [F-19]fluoro or [F-18]fluoro.

2. The compound according to claim 1, wherein 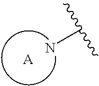
is selected from

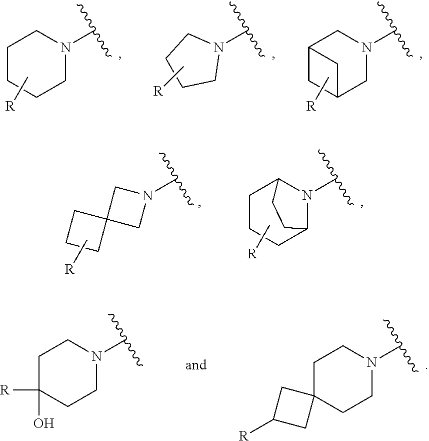

3. The compound according to claim 1, wherein R is selected from F—, F—CH₂CH₂OCH₂CH₂—, F—CD₂CH₂CH₂O—, F—CH₂CH₂O—, F—(CH₂CH₂O)₂—, F—CH₂CH₂O—CH₂—, F—(CH₂CH₂O)₂—CH₂—, F—CH₂CH₂CH₂O—, F—CH₂CH(OH)CH₂O—, F—CH₂CH(F)CH₂O—, HO—CH₂CH(F)CH₂O— and F—CH₂—CH(CH₂OH)—O—.

4. The compound according to claim 1, wherein if R is attached to a N-atom, R is selected from F—CH₂CH₂—, F—CH₂CH₂OCH₂CH₂—, F—CH₂CH₂CH₂—, F—CH₂CH(OH)CH₂— and HO—CH₂CH(F)CH₂—.

5. The compound according to claim 1, wherein the compound is selected from a compound of formula (IA), (IB), (IC), (ID), (IE), (IF), (IG), (IH) and (IJ): 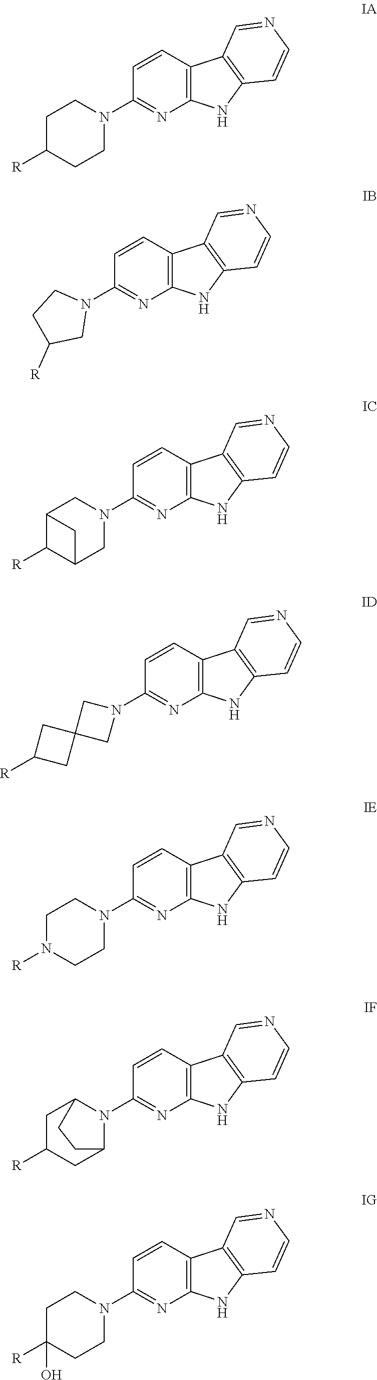 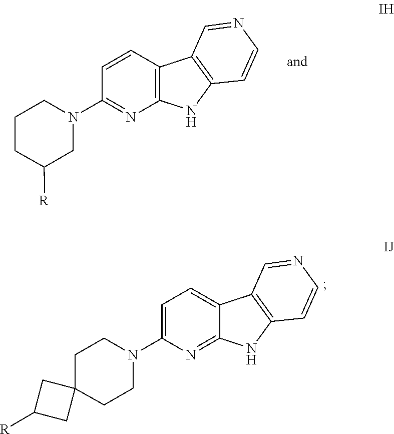
wherein R is as defined in any one of claim 1, 3, or 4.

6. A compound of formula (II): 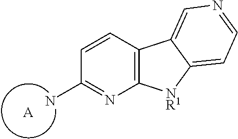
and all stereoisomers and mixtures thereof, pharmaceutically acceptable salts, hydrates, solvates and polymorphs thereof;
wherein 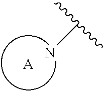
is selected from a 5- to 7-membered monocyclic group and a 7- to 9-membered bicyclic group, which can optionally contain a heteroatom selected from N and O in addition to the nitrogen atom which is attached to the tricyclic group and wherein 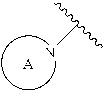
is substituted by a substituent R^{p} and is optionally substituted by a substituent R**;
wherein
R^{p} is selected from LG-, alkylene which is substituted by LG, (alkylene-oxy)ₙ- which is substituted by LG, (alkylene-oxy)ₙ-alkylene- which is substituted by LG, and alkylene-oxy which is substituted by LG and OPG²;
R** is selected from hydroxy, OPG², methoxy, methyl, hydroxymethyl and PG²-O-methyl;
n is selected from 1, 2 and 3;
R¹ is —H or PG¹, wherein PG¹ is an amine protecting group;
PG² is a hydroxy protecting group; and
LG is a leaving group.

7. A diagnostic composition comprising a compound according to claim 1 and a pharmaceutically acceptable carrier, diluent, adjuvant or excipient.

8. (canceled)

9. (canceled)

10. (canceled)

11. (canceled)

12. A method of imaging of tau aggregates comprising administering an effective amount of a compound according to claim 1 to a patient in need thereof.

13. A method of diagnosing a disorder associated with tau aggregates or a tauopathy comprising administering an effective amount of a compound according to claim 1 to a patient in need thereof.

14. The method according to claim 13, wherein the disorder is selected from Alzheimer's disease (AD), familial AD, Creutzfeldt-Jacob disease, dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis, Parkinsonism-dementia complex of Guam, non-Guamanian motor neuron disease with neurofibrillary tangles, argyrophilic grain disease, corticobasal degeneration, diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease, progressive subcortical gliosis, progressive supranuclear palsy (PSP), subacute sclerosing panencephalitis, tangle only dementia, postencephalitic Parkinsonism, myotonic dystrophy, tau panencephalopathy, AD-like with astrocytes, certain prion diseases (GSS with tau), mutations in LRRK2, Hallervorden-Spatz disease, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, and white matter tauopathy with globular glial inclusions; preferably Alzheimer's disease.

15. A method of preparing a compound of formula (I) as defined in claim 1, comprising reacting a compound of formula (II) 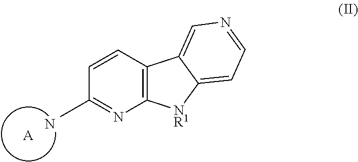
including any stereoisomers or mixtures thereof, pharmaceutically acceptable salts, hydrates, solvates and polymorphs thereof;
and wherein 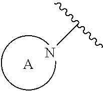
is selected from a 5- to 7-membered monocyclic group and a 7- to 9-membered bicyclic group, which can optionally contain a heteroatom selected from N and O in addition to the nitrogen atom which is attached to the tricyclic group and wherein 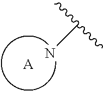
is substituted by a substituent R^{p} and is optionally substituted by a substituent R**;
wherein
R^{p} is selected from LG-, alkylene which is substituted by LG, (alkylene-oxy)ₙ- which is substituted by LG, (alkylene-oxy)ₙ-alkylene- which is substituted by LG, and alkylene-oxy which is substituted by LG and OPG²;
R** is selected from hydroxy, OPG², methoxy, methyl, hydroxymethyl and PG²-O-methyl;
n is selected from 1, 2 and 3;
R¹ is —H or PG¹, wherein PG¹ is an amine protecting group;
PG² is a hydroxy protecting group; and
LG is a leaving group.
with an ¹⁸F-fluorinating agent, so that the leaving group LG is replaced by ¹⁸F.

16. The method of claim 12, wherein the imaging comprises positron emission tomography.

17. The method of claim 15, further comprising cleaving of the protecting group PG¹ or PG², if present
